(19) United States
(12) Patent Application Publication
(10) Pub. No.: US 2004/0072806 A1
(43) Pub. Date: Apr. 15, 2004

(54) Neuroprotective spirostenol pharmaceutical compositions

(76) Inventors: Zhi-Xing Yao, Arlington, VA (US); Laurent Lecanu, McLean, VA (US); Gary L. Teper, Potomac, MD (US); Janet Greeson, Las Vegas, NV (US); Vassilios Papadopoulos, North Potomac, MD (US)

Correspondence Address: MAYER, BROWN, ROWE & MAW, P.O. BOX 2828, CHICAGO, IL, 60690, US

(21) Appl. No.: 10/389,189

(22) Filed: Mar. 14, 2003

Related U.S. Application Data
(60) Provisional application No. 60/364,140, filed on Mar. 15, 2002.
(60) Provisional application No. 60/319,846, filed on Jan. 9, 2003.

(51) Int. Cl. A61K 31/56

(52) U.S. Cl. 514/169; 514/177; 514/178

(57) ABSTRACT

The present invention relates to methods, kits, combinations, and compositions for treating, preventing or reducing the risk of developing a disorder or disease related to, or the symptoms associated with, neurotoxicity in a subject, particularly to beta-amyloid-induced neurotoxicity and Alzheimer's disease. The compounds of the present invention are biologically active 22R-hydroxycholesterol derivatives containing a common spirost-5-en-3-ol structure. The invention further provides a method for treating a patient having a neurological disease or disorder such as global and focal ischemic and hemorrhagic stroke, head trauma, spinal cord injury, hypoxia-induced nerve cell damage, nerve cell damage caused by cardiac arrest or neonatal distress, epilepsy, anxiety, diabetes mellitus, multiple sclerosis, phantom limb pain, causalgia, neuralgias, herpes zoster, spinal cord lesions, hyper algesia, allodynia, Huntington's disease, and Parkinson's disease, by administering to the patient a therapeutically effective amount of 22R-hydroxycholesterol or a therapeutically active analog thereof.

### RELATED APPLICATIONS DATA

This application claims priority to U.S. Provisional Patent Application No. 60/364,140, filed Mar. 15, 2002, and U.S. Provisional Patent Application No. 60/319,846, filed Jan. 9, 2003, both of which are incorporated herein by reference.

### FIELD OF THE INVENTION

The present invention relates to a novel method of prevention or treatment of diseases where deposits of β-amyloid induce cytotoxicity. More particularly, the present invention relates to a pharmaceutical composition comprising a spirostenol, to methods of treatment comprising administering such a pharmaceutical composition to a subject in need thereof, a method for the manufacture of such a composition, to the use of such a composition in treating disease, to combinations with such a composition with other therapeutic agents, and to kits containing such a composition.

### BACKGROUND

Nerve cell death (degeneration) can cause potentially devastating and irreversible effects for an individual and may occur for example, as a result of stroke, heart attack or other brain or spinal chord ischemia or trauma. Additionally, neurodegenerative disorders that involve nerve cell death include Alzheimer's disease, Parkinson's disease, Huntington's disease, Amyotrophic Lateral Sclerosis, Down's Syndrome and Korsakoff's disease.

Alzheimer's Disease (AD) is a progressive neurodegenerative disorder characterized clinically by progressive loss of intellectual function. AD affects about 10% of the population who are beyond the age 65 It attacks 19% of individuals 75 to 85 years old, and 45% of individuals over age 85. AD is the fourth leading cause of death in adults, behind heart disease, cancer, and stroke. AD accounts for about 75% of senile dementia. This central nervous system disorder is marked by a variety of symptoms such as degeneration of neurons, development of amyloid plaques, neurofibrillary tangles, declination of acetylcholine, and atrophy of cerebral cortex. Patients with AD suffer loss of short-term memory initially followed by a decline in cognitive function and finally a loss of the ability to care for themselves. The cost of caring for patients, including diagnosis, nursing, at-home care, and lost wages is estimated at between about $80 billion and $90 billion per year.

The drastic impairment of function associated with AD is caused by the presence of neuritic plaques in the neocortex and hippocampus and the loss of presynaptic markers of cholinergic neurons. Neuritic plaques are composed of degenerating axons and nerve terminals, often surrounding an amyloid core and usually containing reactive glial elements. Another characteristic pathologic feature of Alzheimer's Disease is the neurofibrillary tangle, which is an intraneuronal mass, which corresponds to an accumulation of abnormally phosphorylated tau protein polymerized into fibrillar structures termed paired helical filaments. In addition, the neurofibrillary tangle also contains highly phosphorylated neurofilament proteins.

Although there has been significant progress in unfolding the pathophysiologic mechanisms of the disease, the cause of AD is still poorly understood. There are several suspected causes, such as genetic predisposition (PS-1, PS-2, APP, apoE, CO1, CO2 gene mutations), neurotransmitter defects (acetylcholine deficiency), inflammation, metabolic decline, free radical stress, or excitatory amino acid toxicity.

Several compounds are currently under clinical studies for the treatment of AD according to the current understanding of its pathogenesis. Among these drugs notably are acetylcholine esterase (AchE) inhibitors. Recently, two AchE inhibitors, tacrine and donepezil, have received regulatory approval for AD treatment. While tacrine provides a moderate beneficial effect on deterioration of cognition, it suffers some adverse effects as it causes increases in serum hepatic enzymes.

It thus would be highly desirable to have new neuroprotective agents, particularly agents to limit the extent or otherwise treat nerve cell death (degeneration) such as may occur with stroke, heart attack or brain or spinal cord trauma, or to treat neurodegenerative disorders such as Alzheimer's disease, Parkinson's disease, Huntington's disease, Amyotrophic Lateral Sclerosis, Down's Syndrome and Korsakoff's disease.

Alzheimer's disease is characterized by the accumulation of a 39-43 amino acid peptide termed the β-amyloid protein or Aβ, in a fibrillar form, existing as extracellular amyloid plaques, and as amyloid within the walls of cerebral blood vessels. Fibrillar Aβ amyloid deposition in Alzheimer's disease is believed to be detrimental to the patient and eventually leads to toxicity and neuronal cell death, characteristic hallmarks of AD. Accumulating evidence implicates amyloid as a major causative factor of AD pathogenesis.

A variety of other human diseases also demonstrate amyloid deposition and usually involve systemic organs (i.e., organs or tissues lying outside the central nervous system), with the amyloid accumulation leading to organ dysfunction or failure. In AD and “systemic” amyloid diseases, there is currently no cure or effective treatment, and the patient usually dies within 3 to 10 years from disease onset.

Aβ, which is produced by proteolytic cleavage of β-amyloid precursor protein, is a major component of senile plaques and cerebrovascular angiopathy. Genetic, biochemical as well as histological studies strongly implicated Aβ in the pathogenesis of AD, which is clinically characterized by progressive cognitive impairment and memory deficit. Selkoe, D. J (1999) Nature 399, A23-31; Yankner, B. A. (1996) Neuron 16, 921-932; Selkoe, D. J. (1989) Cell 58, 611-612; Kalaria, R. N. (1996) Pharmacol. Ther. 72,193-214.

Much work in AD has been accomplished, but little is conventionally known about compounds or agents for therapeutic regimes to arrest amyloid formation, deposition, accumulation and/or persistence that occurs in AD and other amyloidoses.

New compounds or agents for therapeutic regimes to arrest or reverse amyloid formation, deposition, accumulation and/or persistence that occurs in AD and other amyloidoses are therefore needed.

Consequently, it would be greatly beneficial if new therapies could be designed based on identified existing compounds, rationally modified compounds and/or de novo designed compounds which are active as Aβ functional inhibitors.

### SUMMARY OF THE INVENTION

The present invention is directed to methods, kits, combinations, and compositions for treating, preventing or reducing the risk of developing a disorder or disease related to, or the symptoms associated with, neurotoxicity in a subject, particularly to beta-amyloid-induced neurotoxicity. The compounds of the present invention are biologically active 22R-hydroxycholesterol derivatives containing a common spirost-5-en-3-ol structure, and having the structure of formula (I), disclosed below.

The present invention is directed to a method of treating a condition or disorder where treatment with a neurotoxicity inhibiting agent of formula (I) is indicated, the method comprises administration of a composition of the present invention to a subject in need thereof. More specifically, the subject invention provides a method for inhibiting the neurotoxic effects of Aβ formation or persistence of brain β-amyloid deposits in a patient, the method comprising administering to the patient a therapeutically effective amount of a compound of formula (I).

In one aspect, the invention provides a method for promoting, maintaining or enhancing in a patient one or more of the mental or cognitive qualities selected from the group of mental or cognitive qualities associated with β-amyloid formation consisting of memory, concentration, and short term memory, the method comprising administering to the patient a therapeutically effective amount of a compound of formula (I).

In another aspect, the invention provides a method for reducing in a patient one or more of the mental or cognitive effects associated with β-amyloid formation selected from the group of mental or cognitive effects associated with β-amyloid formation consisting of cognitive or memory decline and mental decline, the method comprising administering to the patient a therapeutically effective amount of a compound of formula (I).

In yet another aspect, the invention provides a method for treating in a patient mental states associated with β-amyloid formation or persistence, the method comprising administering to the patient a therapeutically effective amount of a compound of formula (I).

In still another aspect the invention provides a method for treating a patient having a neurological disease or disorder selected from the group consisting of global and focal ischemic and hemorrhagic stroke, head trauma, spinal cord injury, hypoxia-induced nerve cell damage, nerve cell damage caused by cardiac arrest or neonatal distress, epilepsy, anxiety, diabetes mellitus, multiple sclerosis, phantom limb pain, causalgia, neuralgias, herpes zoster, spinal cord lesions, hyperalgesia, allodynia, AD, Huntington's disease, and Parkinson's disease, wherein said treatment comprises administering to the patient a therapeutically effective amount of a compound of formula (I).

In a further aspect, the invention provides a method for treating a disease characterized by β-amyloid deposits in the heart, spleen, kidney, adrenal cortex, or liver of a patient comprising administering to the patient a therapeutically effective amount of a compound of formula (I).

In a still further aspect, the invention provides a method of identifying a compound having binding affinity to β-amyloid comprising screening a database of known chemical compounds for structural homology to 22R-hydroxycholesterol; ranking the compounds in the database based on the degree of homology to 22R-hydroxycholesterol, extracting from the database compounds having the highest structural homology to 22R-hydroxycholesterol; ranking the extracted compounds according to in vitro binding to β-amyloid; and selecting the compound having the highest in vitro affinity.

In still another aspect, the invention provides a method of designing a compound having binding affinity to β-amyloid comprising mapping 22R-hydroxycholesterol into two or more separate building blocks; designing a new compound by modifying one or more blocks of 22R-hydroxycholesterol, ranking the designed compound according to in vitro binding to β-amyloid; and selecting the compound having the highest in vitro binding affinity.

In a further aspect, the invention provides a method of designing a compound having binding affinity to β-amyloid comprising mapping β-amyloid, constructing on a computer screen a compound that complements the structure of β-amyloid or a fragment thereof; ranking the designed compound according to in vitro binding to β-amyloid; and selecting the compound having the highest in vitro binding affinity.

In yet another aspect, the invention provides a method of detection and quantification of Aβ in biological fluid comprising obtaining a sample fluid; incubating the fluid with labeled compound of formula (I); optionally in the presence of increasing concentrations of unlabeled compound; separating samples from the incubation fluid and transferring the samples to a nitrocellulose membrane; exposing the membrane to tritium-sensitive screen; and analyzing the contents of the membrane by phospho-imaging to detect the presence of Aβ or quantifying the amount of Aβ present in the biological fluid.

In still another aspect, the invention provides a method of diagnosing AD in a subject comprising obtaining a sample fluid from the brain of the subject; incubating the fluid with labeled compound of formula (I); optionally in the presence of increasing concentrations of unlabeled compound; separating samples from the incubation fluid and transferring the samples to a nitrocellulose membrane; exposing the membrane to tritium-sensitive screen; and analyzing the contents of the membrane by phospho-imaging to detect the presence of Aβ or quantifying the amount of Aβ present in the biological fluid.

Accordingly, a principal aspect of this invention relates to a pharmaceutical composition for treating a disorder related to a beta-amyloid-induced neurotoxicity or a neurodegenerative disorder in a subject. This composition includes an effective amount of a compound of formula (I) and a pharmaceutically acceptable carrier. Also within the scope of this invention is the use of a compound of formula (I) for the manufacture of a medicament to be used in treating one of such disorders. Treatment of these conditions is accomplished by administering to a subject a therapeutically effective amount of a compound or composition of the present invention.

The details of one or more embodiments of the invention are set forth in the accompanying description below. Other features, objects, and advantages of the invention will be apparent from the description and claims.

### BRIEF DESCRIPTION OF THE DRAWINGS

The figures illustrate some of the compounds of the invention, methods for identifying those compounds and results of in vitro and in vivo biological test demonstrating the activity of illustrative compounds according to the invention.

FIG. 1 illustrates several of the structures of the chemical structure of 22R-hydroxycholesterol (SP222) and naturally occurring derivatives.

FIG. 2 is a chart describing 22R-hydroxycholesterol levels in AD and control brain specimens.

FIG. 3A is a line graph depicting the effect of increasing concentrations of 22R-hydroxycholesterol on rat PC12 neuronal cell viability in the absence or presence of increasing concentration of Aβ₁₋₄₂.

FIG. 3B is a line graph depicting the effect of increasing concentrations of cholesterol on rat PC12 neuronal cell viability in the absence or presence of increasing concentration of Aβ₁₋₄₂.

FIG. 3C is a line graph depicting the effect of increasing concentrations of pregnenolone on rat PC12 neuronal cell viability in the absence or presence of increasing concentration of Aβ₁₋₄₂.

FIG. 3D is a line graph depicting the effect of increasing concentrations of 17α-hydroxypregnenolone on rat PC12 neuronal cell viability in the absence or presence of increasing concentration of Aβ₁₋₄₂.

FIG. 3E is a line graph depicting the effect of increasing concentrations of DHEA on rat PC12 neuronal cell viability in the absence or presence of increasing concentration of Aβ₁₋₄₂.

FIG. 3F is a line graph depicting the effect of increasing concentrations of 22S-hydroxycholesterol on rat PC12 neuronal cell viability in the absence or presence of increasing concentration of Aβ₁₋₄₂.

FIG. 4 is a line graph depicting the effect of 22R-hydroxycholesterol on differentiated human NT2N neuron viability determined in absence or presence of Aβ₁₋₄₂.

FIG. 5A is a line graph depicting the effect of 22R-hydroxycholesterol and DHEA on Aβ₁₋₄₂-induced toxicity on rat PC12 neuronal cells.

FIG. 5B is a line graph depicting the effect of 22R-hydroxycholesterol and DHEA on Aβ₂₅₋₃₅-induced toxicity on rat PC12 neuronal cells.

FIG. 5C is a line graph depicting the effect of 22R-hydroxycholesterol and DHEA on Aβ₁₋₄₂-induced toxicity on human NT2 cells.

FIG. 5D is a line graph depicting the effect of 22R-hydroxycholesterol and DHEA on Aβ₂₅₋₃₅-induced toxicity on human NT2 cells.

FIG. 6A is a coomassie blue gel depicting the effect of 22R-hydroxycholesterol on Aβ aggregation.

FIG. 6B is an immunoblot analysis of the coomassie blue stained gel of FIG. 6A depicting the effect of 22R-hydroxycholesterol on Aβ aggregation.

FIG. 7A is an immunoblot analysis identifying Aβ₁₋₄₂-22R-hydroxycholesterol binding and binding site by CPBBA.

FIG. 7B is an immunoblot analysis identifying Aβ₁₋₄₂ by a polyclonal rabbit anti-β-amyloid peptide antiserum on the blot shown in FIG. 7A.

FIG. 7C is an immunoblot analysis identifying the 22R-hydroxycholesterol binding site on Aβ.

FIG. 7D is a computational 22R-hydroxycholesterol docking simulation to Aβ₁₋₄₂.

FIG. 7E is a computational 22R-hydroxycholesterol docking simulation to Aβ₁₇₋₄₀.

FIG. 7F is a computational 22R-hydroxycholesterol docking simulation to Aβ₁₇₋₄₀.

FIG. 7G is a computational 22R-hydroxycholesterol docking simulation to Aβ₁₋₄₂.

FIG. 7H is a computational 22R-hydroxycholesterol docking simulation to Aβ₁₇₋₄₀.

FIG. 7I is an amino acid sequence of the localization of the 22R-hydroxycholesterol binding site in Aβ₁₋₄₂.

FIG. 8 is a bar graph illustrating that three days' exposure of PC12 cells to increasing concentrations of Aβ resulted in dose-dependent cell death.

FIGS. 9A to 9P are a series of bar graphs illustrating the effect increasing concentrations of 22R-hydroxycholesterol (SP222) and derivatives on rat PC12 neuronal cell viability in the absence or presence of 0.1 μM of Aβ₁₋₄₂.

FIGS. 10A to 10P are a series of bar graphs illustrating the effect increasing concentrations of 22R-hydroxycholesterol (SP222) and derivatives on rat PC12 neuronal cell viability in the absence or presence of 1.0 μM of Aβ₁₋₄₂.

FIGS. 11A to 11P are a series of bar graphs illustrating the effect increasing concentrations of 22R-hydroxycholesterol (SP222) and derivatives on rat PC12 neuronal cell viability in the absence or presence of 10.0 μM of Aβ₁₋₄₂.

FIG. 12A is a bar graph showing that Aβ exposure induces a dose-related decrease of the membrane potential-assessing luminescence.

FIG. 12B is a bar graph showing the effect of 22R-hydroxycholesterol (SP222) and derivatives against 0.1 μM Aβ-induced neurotoxicity.

FIG. 12C is a bar graph showing the effect of 22R-hydroxycholesterol (SP222) and derivatives against 1.0 μM Aβ-induced neurotoxicity.

FIG. 12D is a bar graph showing the effect of 22R-hydroxycholesterol (SP222) and derivatives against 10.0 μM Aβ-induced neurotoxicity.

FIG. 13A is a bar graph showing that Aβ decreased in a dose-dependent manner ATP production by PC12 cells in the presence of 0.1, 1.0 and 10.0 μM Aβ-induced neurotoxicity.

FIG. 13B is a bar graph showing the effect of 22R-hydroxycholesterol (SP222) and derivatives on ATP in the presence of 0.1 μM Aβ-induced neurotoxicity.

FIG. 13C is a bar graph showing the effect of 22R-hydroxycholesterol (SP222) and derivatives on ATP in the presence of 1.0 μM Aβ-induced neurotoxicity.

FIG. 13D is a bar graph showing the effect of 22R-hydroxycholesterol (SP222) and derivatives on ATP in the presence of 10.0 μM Aβ-induced neurotoxicity.

FIG. 14A is a line graph showing trypan blue uptake by cells in the presence of Aβ alone; Aβ+SP233 30 μM; and Aβ+SP233 50 μM.

FIG. 14B is a line graph showing the effect of increasing concentrations of SP233 on 0.1, 1.0, and 10.0 μM Aβ-induced neurotoxicity on rat PC12 neuronal cell

FIG. 15 is a line graph illustrating the effect of SP233 on MA-10 Leydig cell steroid formation.

FIG. 16 is a bar graph identifying Aβ-SP binding and binding site by CPBBA.

FIGS. 17A-17Q are computational docking simulations of the compounds of Table 1 to Aβ₁₋₄₂.

FIG. 18A is a computational docking simulation depicting the binding energy frequencies of 22R-hydroxycholesterol (SP222) and SP233 to Aβ₁₋₄₂.

FIG. 18B is a computational docking simulation depicting the probabilities of 22R-hydroxycholesterol (SP222) and SP233 binding to Aβ₁₋₄₂.

FIG. 19 is computer simulation of the basic spirostenol structure present in the neuroprotective SP compounds.

### DETAILED DESCRIPTION OF THE INVENTION

While the present invention may be embodied in many different forms, several specific embodiments are discussed herein with the understanding that the present disclosure is to be considered only as an exemplification of the principles of the invention, and it is not intended to limit the invention to the embodiments illustrated.

Abbreviations used herein are as follows: 5-cholesten-3β,22R-diol, (22R-hydroxycholesterol); 5-cholesten-3β, 22S-diol, (22S-hydroxycholesterol); 5-cholesten-3β-ol (cholesterol); 5-androsten-3β-ol-17-one or dehydroepiandrosterone (DHEA); 5-pregnen-3β,17α-diol-20-one (17α-hydroxypregnenolone); 5-pregnen-3β-ol-20-one (pregnenolone); Ntera2/D1 teratocarcinoma cells (NT2); differentiated human NT2 neurons (NT2N); β-amyloid peptide, (Aβ); Alzheimer's disease, (AD); cholesterol-protein binding blot assay (CPBBA).

The present invention is based on the unexpected discovery that 22R-hydroxycholesterol, a steroid intermediate in the pathway of pregnenolone formation from cholesterol, is present at lower levels in AD hippocampus and frontal cortex tissue specimens compared to age-matched controls. As discussed above, Amyloid β (Aβ) peptide has been shown to be neurotoxic and its presence in the brain has been linked to AD pathology.

As described below, the present inventors have unexpectedly discovered that 22R-hydroxycholesterol protects, in a dose-dependent manner, against Aβ-induced rat sympathetic nerve pheochromocytoma (PC12) and differentiated human NT2N neuronal cell death. The effect of 22R-hydroxycholesterol was found to be stereospecific because its enantiomer 22S-hydroxycholesterol failed to protect the neurons from Aβ-induced cell death. Such rat models have general applicability to humans.

One aspect of this invention relates to a method of treating a disorder related to neurotoxicity, particularly AD, comprising administering to a subject in need thereof a compound of formula (I):

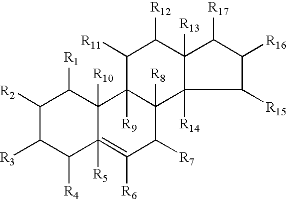

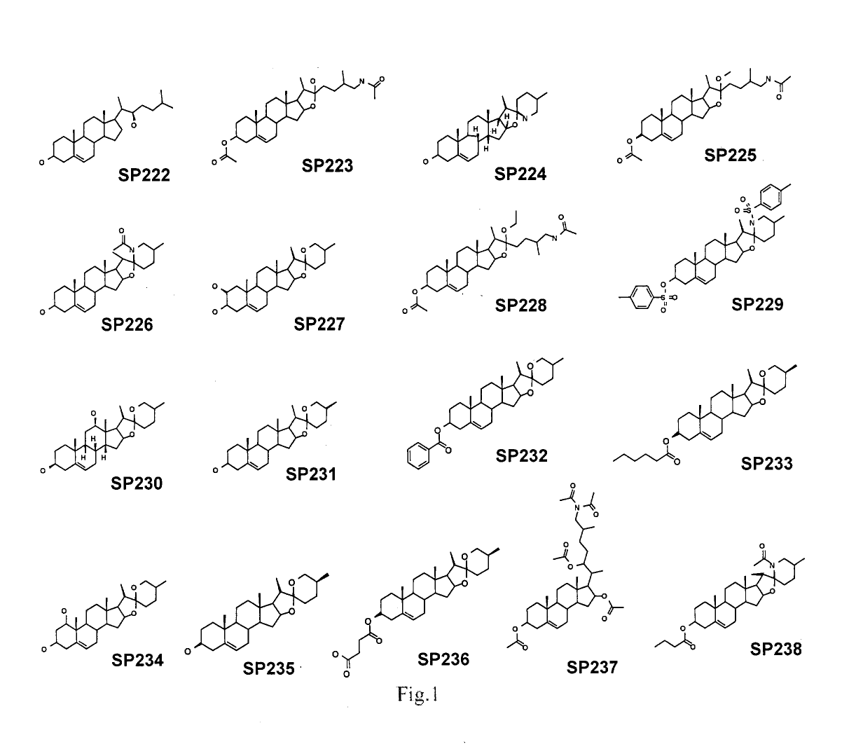

In formula (I), each of R₁, R₂, R₄, R₅, R₆, R₇, R₁₁, R₁₂, R₁₅, and R₁₆, independently, is hydrogen, alkyl, hydroxy, amino, carboxyl, oxo, sulfonic acid, or alkyl that is optionally inserted with —NH—, —N(alkyl)-, —O—, —S—, —SO—, —SO₂—, —O—SO₂—, —SO₂—O—, —SO₃—O—, —CO—, —CO—O—, —O—CO—, —CO—NR′—, or —NR′—CO-—R₃ is a substituent as disclosed at R₃ of the compounds listed in Table 1 and FIG. 1; each of R₈, R₉, R₁₀, R₁₃, and R₁₄, independently, is hydrogen, alkyl, hydroxyalkyl, alkoxy, or hydroxy; and R₁₇ is a substituent as disclosed at R₁₇ of the compounds listed in Table 1 and FIG. 1. Note that the carbon atoms shown in formula (I) are saturated with hydrogen unless otherwise indicated.

Each of the term “alkyl,” the prefix “alk” (as in alkoxy), and the suffix “-alkyl” (as in hydroxyalkyl) refers to a C₁₋₈ hydrocarbon chain, linear (e.g., butyl) or branched (e.g., iso-butyl). Alkylene, alkenylene, and alkynylene refer to divalent C₁₋₈ alkyl (e.g., ethylene), alkene, and alkyne radicals, respectively.

Unless otherwise defined, all technical and scientific terms used herein have the same meaning as commonly understood by one of ordinary skills in the art to which this invention belongs.

Shown below in Table 1 are several compounds of formula (I) described above that can be used to practice this invention:

TABLE 1
Chemical name, denomination and origin of naturally occurring
compounds containing the 22R-hydroxycholesterol lead structure.

| Denomination | Chemical Name | Orgin |
|---|---|---|
| SP222 | 22R-hydroxycholesterol | Mammalian |
| SP223 | (20ξ)-26-acetylamino-(22ξ)-hydroxyfurost-5-en-3ξ-yl acetate | Gynura sp. (asteraceae) |
| SP224 | (20α)-25ξ-methyl-(22R,26)-azacyclofurost-5-en-3ξ-ol | Solanum asperum |
| | | (solanaceae) |
| SP225 | (20ξ)-26-acetylamino-(22ξ)-methoxyfurost-5-en-3α-yl acetate | Gynura sp. (asteraceae) |
| SP226 | (20ξ)-25ξ-methyl-N-acetyl-(22R,26)-azacyclofurost-5-en-3ξ-ol | Solanum asperum |
| | | (solanaceae) |
| SP227 | (22R,25ξ)-(20α)-spirost-5-en-(2α,3ξ)-diol | Gynura japonica |
| | | (asteraceae) |
| SP228 | (20ξ)-26-acetylamino-(22ξ)-ethoxyfurost-5-en-3ξ-yl acetate | Gynura sp. (asteraceae) |
| SP229 | (20α)-25ξ-methyl-N-paratoluenesulfonyl-(22R,26)- | Solanum aviculare |
| | azacyclofurost-5-en-3ξ-yl paratoluenesulfonate | (solanaceae) |
| SP230 | (22R,25ξ)-(20α)-(14α,20α)-spirost-5-en-(3β,12β)-diol | Gynura japonica |
| | | (asteraceae) |
| SP231 | (22R,25S)-(20ξ)-spirost-5-en-3ξ-ol | Gynura japonica |
| | | (asteraceae) |
| SP232 | (22R,25ξ)-(20α)-spirost-5-en-3β-yl benzoate | Gynura sp. (asteraceae) |
| SP233 | (22S,25S)-(20S)-spirost-5-en-3β-yl hexanoate | Gynura sp. (asteraceae) |
| SP234 | (22R,25ξ)-(20α)-spirost-5-en-(1ξ,3ξ)-diol | Gynura japonica |
| | | (asteraceae) |
| SP235 | (22R,25S)-(20α)-spirost-5-en-3β-ol | Gynura japonica |
| | | (asteraceae) |
| SP236 | (22R,25S)-(20α)-spirost-5-en-3β-yl succinate | Gynura sp. (asteraceae) |
| SP237 | 26-diacetylamino-(22ξ)-acetoxy-(16ξ)-acetoxy-cholest-5-en-yl | Achlya heterosexualis |
| | acetate | (saprolegniaceae) |
| SP238 | (20α)-25S-methyl-N-acetyl-(22S,26)-azacyclofurost-5-en-3β-yl | Solanum asperum |
| | propanoate | (solanaceae) |

Other 22R-hydroxycholesterol derivatives may be identified through structure-based database searching. Two approaches may be followed. One approach is based on the structure of 22R-hydroxycholesterol. 22R-hydroxycholesterol is subdivided into several building blocks, the database is searched for compounds that include one or more of the building blocks of 22R-hydroxycholesterol. A refined search based on the results presented in this application may be formulated such that the 22R hydroxy functionality of the 22R-hydroxycholesterol is conserved. Compounds having structural similarity to 22R-hydroxycholesterol are extracted from the database and tested in vitro for their binding affinity to Aβ. The compounds with the highest binding affinity are selected for further in vivo studies. The second approach is based on the structure of Aβ. Briefly, in (receptor) structure-based 3D-database searching, the 3D structure of the target molecule Aβ is determined through NMR analysis, then large chemical databases containing the 3D structures of hundreds of thousands of structurally diverse synthetic compounds and natural products are searched through computerized molecular docking to identify small molecules that can interact effectively with Aβ.

In forming a template 3-D structure of Aβ, each atom of the backbone of the Aβ is assigned a position according to a starting conformation, the positions for the atoms of the side chains are assigned according to the internal coordinates of minimum energy for each side chain. The template structure thus obtained is refined by minimizing the internal energy of the template. Based on the refined structure of Aβ, a host-guest complex is formed by disposing a compound from a compound database around Aβ. The structure of the host-guest complex is defined by the position occupied by each atom in the complex in a three dimensional referential.

A geometry-fit group is formed by selecting the compounds which can be disposed in the target binding site without significant unfavorable overlap with the atoms of the Aβ. For each compound in the geometry fit group, a predicted binding affinity to the receptor site of Aβ is determined by minimizing an energy function describing the interactions between the atoms of the compound and those of Aβ. The minimization of the energy function is conducted by changing the position of the compound such that a guest-host complex structure corresponding to a minimum of the energy function is obtained. The compounds having the most favorable energy interaction with the atoms of the binding site are identified for optional further processing, for example through display and visual inspection of compound Aβ complexes to identify the most promising compound candidates.

The displayed complexes are visually examined to form a group of candidate compounds for in vitro testing. For example, the complexes are inspected for visual determination of the quality of docking of the compound into the receptor site of Aβ. Visual inspection provides an effective basis for identifying compounds for in vitro testing.

After putative binding compounds have been identified, the ability of such compounds to specifically bind to Aβ is confirmed in vitro and/or in vivo.

In another aspect, the present invention provides novel compounds which are rationally designed to inhibit to bind to Aβ. Rational design of the novel compounds is based on information relating to the binding site of Aβ. The structures of Aβ and a lead compound is analyzed such that compound structures having possible activity in binding to the binding site of Aβ are formulated.

The structure of the lead compounds is divided into design blocks, the modification of which is probed for influence on the interactions between the lead compound and the binding site of Aβ. Compounds having different design block combinations are then synthesized and their activity in relation to the identified mechanism is tested. Such tests are conducted in vitro and/or in vivo, in the same manner described above. The information obtained through such tests is then incorporated in a new cycle of rational drug design. The design-synthesis-testing cycle is repeated until a lead compound having the desired properties is identified. The lead compound is then clinically tested.

The term “treat” or “treatment” as used herein refers to any treatment of a disorder or disease associated with a disease or disorder related to neurotoxicity, or beta-amyloid-induced neurotoxicity, in a subject, and includes, but is not limited to, preventing the disorder or disease from occurring in a subject who may be predisposed to the disorder or disease, but has not yet been diagnosed as having the disorder or disease; inhibiting the disorder or disease, for example, arresting the development of the disorder or disease; relieving the disorder or disease, for example, causing regression of the disorder or disease; or relieving the condition caused by the disease or disorder, for example, stopping the symptoms of the disease or disorder. As used herein, “neurodegenerative disorder” is intended to encompass all disorders stated above.

The term “prevent” or “prevention,” in relation to a disease or disorder related to neurotoxicity, or beta-amyloid-induced neurotoxicity, in a subject, means no disease or disorder development if none had occurred, or no further disorder or disease development if there had already been development of the disorder or disease.

An effective amount of an efficacious compound can be formulated with a pharmaceutically acceptable carrier to form a pharmaceutical composition before being administered for treatment of a disease related to neurotoxicity. “An effective amount” or “pharmacologically effective amount” refers to the amount of the compound which is required to confer therapeutic effect on the treated subject. The interrelationship of dosages for animals and humans (based on milligrams per square meter of body surface) is described by Freireich et al., Cancer Chemother. Rep., 1966, 50, 219. Body surface area may be approximately determined from height and weight of the patient. See, e.g., Scientific Tables, Geigy Pharmaceuticals, Ardley, N.Y., 1970, 537. Effective doses will also vary, as recognized by those skilled in the art, depending on the route of administration, the excipient usage, and the optional co-administration with other therapeutic agents.

Toxicity and therapeutic efficacy of the active ingredients can be determined by standard pharmaceutical procedures, e.g., for determining LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Included in the methods, kits, combinations and pharmaceutical compositions of the present invention are the crystalline forms (e.g., polymorphs), isomeric forms and tautomers of the described compounds and the pharmaceutically-acceptable salts thereof. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

The term “prodrug” refers to a drug or compound (active moeity) that elicits the pharmacological action results from conversion by metabolic processes within the body. Prodrugs are generally considered drug precursors that, following administration to a subject and subsequent absorption, are converted to an active or a more active species via some process, such as a metabolic process. Other products from the conversion process are easily disposed of by the body. Prodrugs generally have a chemical group present on the prodrug which renders it less active and/or confers solubility or some other property to the drug. Once the chemical group has been cleaved from the prodrug the more active drug is generated. Prodrugs may be designed as reversible drug derivatives and utilized as modifiers to enhance drug transport to site-specific tissues. The design of prodrugs to date has been to increase the effective water solubility of the therapeutic compound for targeting to regions where water is the principal solvent. For example, Fedorak, et al., Am. J. Physiol, 269:G210-218 (1995), describe dexamethasone-beta-D-glucuronide. McLoed, et al., Gastroenterol., 106:405-413 (1994), describe dexamethasone-succinate-dextrans. Hochhaus, et al., Biomed. Chrom., 6:283-286 (1992), describe dexamethasone-21-sulphobenzoate sodium and dexamethasone-21-isonicotinate. Additionally, J. Larsen and H. Bundgaard, Int. J. Pharmaceutics, 37, 87 (1987) describe the evaluation of N-acylsulfonamides as potential prodrug derivatives. J. Larsen et al., Int. J. Pharmaceutics, 47, 103 (1988) describe the evaluation of N-methylsulfonamides as potential prodrug derivatives. Prodrugs are also described in, for example, Sinkula et al., J. Pharm. Sci., 64:181-210 (1975).

The term “derivative” refers to a compound that is produced from another compound of similar structure by the replacement of substitution of one atom, molecule or group by another. For example, a hydrogen atom of a compound may be substituted by alkyl, acyl, amino, etc., to produce a derivative of that compound.

“Plasma concentration” refers to the concentration of a substance in blood plasma or blood serum.

“Drug absorption” or “absorption” refers to the process of movement from the site of administration of a drug toward the systemic circulation, for example, into the bloodstream of a subject.

“Bioavailability” refers to the extent to which an active moiety (drug or metabolite) is absorbed into the general circulation and becomes available at the site of drug action in the body. “Metabolism” refers to the process of chemical transformations of drugs in the body.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasma half-life” refers to the time required for the plasma drug concentration to decrease by 50% from its maximum concentration.

The use of the term “about” in the present disclosure means “approximately,” and illustratively, the use of the term “about” indicates that dosages outside the cited ranges may also be effective and safe, and such dosages are also encompassed by the scope of the present claims.

The term “measurable serum concentration” means the serum concentration (typically measured in mg, μg, or ng of therapeutic agent per ml, dl, or 1 of blood serum) of a therapeutic agent absorbed into the bloodstream after administration.

The term “pharmaceutically acceptable” is used adjectivally herein to mean that the modified noun is appropriate for use in a pharmaceutical product. Pharmaceutically acceptable cations include metallic ions and organic ions. More preferred metallic ions include, but are not limited to appropriate alkali metal (Group Ia) salts, alkaline earth metal (Group IIa) salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

The compositions of the present invention are usually administered in the form of pharmaceutical compositions. These compositions can be administered by any appropriate route including, but not limited to, oral, nasogastric, rectal, transdermal, parenteral (for example, subcutaneous, intramuscular, intravenous, intramedullary and intradermal injections, or infusion techniques administration), intranasal, transmucosal, implantation, vaginal, topical, buccal, and sublingual. Such preparations may routinely contain-buffering agents, preservatives, penetration enhancers, compatible carriers and other therapeutic or non-therapeutic ingredients.

The present invention also includes methods employing a pharmaceutical composition that contains the composition of the present invention associated with pharmaceutically acceptable carriers or excipients. As used herein, the terms “pharmaceutically acceptable carrier” or “pharmaceutically acceptable excipients” includes any and all solvents, dispersion media, coatings, antibacterial and antifungal agents, isotonic and absorption delaying agents, and the like. The use of such media and agents for ingestible substances is well known in the art. Except insofar as any conventional media or agent is incompatible with the compositions, its use is contemplated. Supplementary active ingredients can also be incorporated into the compositions. In making the compositions of the present invention, the compositions(s) can be mixed with a pharmaceutically acceptable excipient, diluted by the excipient or enclosed within such a carrier, which can be in the form of a capsule, sachet, or other container. The carrier materials that can be employed in making the composition of the present invention are any of those commonly used excipients in pharmaceutics and should be selected on the basis of compatibility with the active drug and the release profile properties of the desired dosage form.

Illustratively, pharmaceutical excipients are chosen below as examples:

(a) Binders such as acacia, alginic acid and salts thereof, cellulose derivatives, methylcellulose, hydroxyethyl cellulose, hydroxypropyl cellulose, magnesium aluminum silicate, polyethylene glycol, gums, polysaccharide acids, bentonites, hydroxypropyl methylcellulose, gelatin, polyvinylpyrrolidone, polyvinylpyrrolidone/vinyl acetate copolymer, crospovidone, povidone, polymethacrylates, hydroxypropylmethylcellulose, hydroxypropylcellulose, starch, pregelatinized starch, ethylcellulose, tragacanth, dextrin, microcrystalline cellulose, sucrose, or glucose, and the like.

(b) Disintegration agents such as starches, pregelatinized corn starch, pregelatinized starch, celluloses, cross-linked carboxymethylcellulose, sodium starch glycolate, crospovidone, cross-linked polyvinylpyrrolidone, croscarmellose sodium, microcrystalline cellulose, a calcium, a sodium alginate complex, clays, alginates, gums, or sodium starch glycolate, and any disintegration agents used in tablet preparations.

(c) Filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

(d) Surfactants such as sodium lauryl sulfate, sorbitan monooleate, polyoxyethylene sorbitan monooleate, polysorbates, polaxomers, bile salts, glyceryl monostearate, Pluronic™ line (BASF), and the like.

(e) Solubilizer such as citric acid, succinic acid, fumaric acid, malic acid, tartaric acid, maleic acid, glutaric acid sodium bicarbonate and sodium carbonate and the like.

(f) Stabilizers such as any antioxidation agents, buffers, or acids, and the like, can also be utilized.

(g) Lubricants such as magnesium stearate, calcium hydroxide, talc, sodium stearyl fumarate, hydrogenated vegetable oil, stearic acid, glyceryl behapate, magnesium, calcium and sodium stearates, stearic acid, talc, waxes, Stearowet, boric acid, sodium benzoate, sodium acetate, sodium chloride, DL-leucine, polyethylene glycols, sodium oleate, or sodium lauryl sulfate, and the like.

(h) Wetting agents such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, or sodium lauryl sulfate, and the like.

(i) Diluents such lactose, starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, or bentonite, and the like.

j) Anti-adherents or glidants such as talc, corn starch, DL-leucine, sodium lauryl sulfate, and magnesium, calcium, or sodium stearates, and the like.

(k) Pharmaceutically compatible carrier comprises acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, or pregelatinized starch, and the like.

Additionally, drug formulations are discussed in, for example, Remington's The Science and Practice of Pharmacy (2000). Another discussion of drug formulations can be found in Liberman, H. A. and Lachman, L., Eds., Pharmaceutical Dosage Forms, Marcel Decker, New York, N.Y., 1980. The tablets or granules comprising the inventive compositions may be film coated or enteric-coated.

Besides being useful for human treatment, the present invention is also useful for other subjects including veterinary animals, reptiles, birds, exotic animals and farm animals, including mammals, rodents, and the like. Mammal includes a primate, for example, a monkey, or a lemur, a horse, a dog, a pig, or a cat. A rodent includes a rat, a mouse, a squirrel, or a guinea pig.

The pharmaceutical compositions of the present invention are useful where administration of an inhibitor of neurotoxicity is indicated. It has been found that these compositions are particularly effective in the treatment of senile cognitive impairment and/or dementia (for example, AD).

For treatment of a neurodegenerative disorder, compositions of the invention can be used to provide a dose of a compound of the present invention in an amount sufficient to elicit a therapeutic response, e.g., reduction of Aβ-induced cytoxicity, for example a dose of about 5 ng to about 1000 mg, or about 100 ng to about 600 mg, or about 1 mg to about 500 mg, or about 20 mg to about 400 mg. Typically a dosage effective amount will range from about 0.0001 mg/kg to 1500 mg/kg, more preferably 1 to 1000 mg/kg, more preferably from about 1 to 150 mg/kg of body weight, and most preferably about 50 to 100 mg/kg of body weight. A dose can be administered in one to about four doses per day, or in as many doses per day to elicit a therapeutic effect. Illustratively, a dosage unit of a composition of the present invention can typically contain, for example, about 5 ng, 50 ng 100 ng, 500 ng, 1 mg, 10 mg, 20 mg, 40 mg, 80 mg, 100 mg, 125 mg, 150 mg, 200 mg, 250 mg, 300 mg, 350 mg, 400 mg, 450 mg, 500 mg, 550 mg, 600 mg, 700 mg, 800 mg, 900 mg, or 1000 mg of a compound of the present invention. The dosage form can be selected to accommodate the desired frequency of administration used to achieve the specified dosage. The amount of the unit dosage form of the composition that is administered and the dosage regimen for treating the condition or disorder depends on a variety of factors, including, the age, weight, sex and medical condition, of the subject, the severity of the condition or disorder, the route and frequency of administration, and this can vary widely, as is well known.

In one embodiment of the present invention, the composition is administered to a subject in an effective amount, that is, the composition is administered in an amount that achieves a therapeutically effective dose of a compound of the present invention in the blood serum of a subject for a period of time to elicit a desired therapeutic effect. Illustratively, in a fasting adult human (fasting for generally at least 10 hours) the composition is administered to achieve a therapeutically effective dose of a compound of the present invention in the blood serum of a subject from about 5 minutes after administration of the composition. In another embodiment of the present invention, a therapeutically effective dose of the compound of the present invention is achieved in the blood serum of a subject at about 10 minutes from the time of administration of the composition to the subject. In another embodiment of the present invention, a therapeutically effective dose of the compound of the present invention is achieved in the blood serum of a subject at about 20 minutes from the time of administration of the composition to the subject. In yet another embodiment of the present invention, a therapeutically effective dose of the compound of the present invention is achieved in the blood serum of a subject at about 30 minutes from the time of administration of the composition to the subject. In still another embodiment of the present invention, a therapeutically effective dose of the compound of the present invention is achieved in the blood serum of a subject at about 40 minutes from the time of administration of the composition to the subject. In one embodiment of the present invention, a therapeutically effective dose of the compound of the present invention is achieved in the blood serum of a subject at about 20 minutes to about 12 hours from the time of administration of the composition to the subject. In another embodiment of the present invention, a therapeutically effective dose of the compound of the present invention is achieved in the blood serum of a subject at about 20 minutes to about 6 hours from the time of administration of the composition to the subject. In yet another embodiment of the present invention, a therapeutically effective dose of the compound of the present invention is achieved in the blood serum of a subject at about 20 minutes to about 2 hours from the time of administration of the composition to the subject. In still another embodiment of the present invention, a therapeutically effective dose of the compound of the present invention is achieved in the blood serum of a subject at about 40 minutes to about 2 hours from the time of administration of the composition to the subject. And in yet another embodiment of the present invention, a therapeutically effective dose of the compound of the present invention is achieved in the blood serum of a subject at about 40 minutes to about 1 hour from the time of administration of the composition to the subject.

In one embodiment of the present invention, a composition of the present invention is administered at a dose suitable to provide a blood serum concentration with a half maximum dose of a compound of the present invention. Illustratively, a blood serum concentration of about 0.01 to about 1000 nM, or about 0.1 to about 750 nM, or about 1 to about 500 nM, or about 20 to about 1000 nM, or about 100 to about 500 nM, or about 200 to about 400 nM is achieved in a subject after administration of a composition of the present invention.

Contemplated compositions of the present invention provide a therapeutic effect as compound of the present invention medications over an interval of about 5 minutes to about 24 hours after administration, enabling once-a-day or twice-a-day administration if desired. In one embodiment of the present invention, the composition is administered at a dose suitable to provide an average blood serum concentration with a half maximum dose of a compound of the present invention of at least about 1 μg/ml; or at least about 5 μg/ml, or at least about 10 μg/ml, or at least about 50 μg/ml, or at least about 100 μg/ml, or at least about 500 μg/ml, or at least about 1000 μg/ml in a subject about 10, 20, 30, or 40 minutes after administration of the composition to the subject.

The amount of therapeutic agent necessary to elicit a therapeutic effect can be experimentally determined based on, for example, the absorption rate of the agent into the blood serum, the bioavailability of the agent, and the potency for treating the disorder. It is understood, however, that specific dose levels of the therapeutic agents of the present invention for any particular subject depends upon a variety of factors including the activity of the specific compound employed, the age, body weight, general health, sex, and diet of the subject (including, for example, whether the subject is in a fasting or fed state), the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for subject administration. Studies in animal models generally may be used for guidance regarding effective dosages for treatment of gastrointestinal disorders or diseases in accordance with the present invention. In terms of treatment protocols, it should be appreciated that the dosage to be administered will depend on several factors, including the particular agent that is administered, the route administered, the condition of the particular subject, etc. Generally speaking, one will desire to administer an amount of the compound that is effective to achieve a serum level commensurate with the concentrations found to be effective in vitro for a period of time effective to elicit a therapeutic effect. Thus, where a compound is found to demonstrate in vitro activity at, for example, a half-maximum effective dose of 200 nM, one will desire to administer an amount of the drug that is effective to provide about a half-maximum effective dose of 200 nM concentration in vivo for a period of time that elicits a desired therapeutic effect, for example, treating a disorder related to high beta-amyloid-induced neurotoxicity and other indicators as are selected as appropriate measures by those skilled in the art. Determination of these parameters is well within the skill of the art. These considerations are well known in the art and are described in standard textbooks.

In order to measure and determine the effective amount of a compound of the present invention to be delivered to a subject, serum compound of the present invention concentrations can be measured using standard assay techniques.

Contemplated compositions of the present invention provide a therapeutic effect over an interval of about 30 minutes to about 24 hours after administration to a subject. In one embodiment compositions provide such therapeutic effect in about 30 minutes. In another embodiment compositions provide therapeutic effect over about 24 hours, enabling once-a-day administration to improve patient compliance.

The present methods, kits, and compositions can also be used in combination (“combination therapy”) with another pharmaceutical agent that is indicated for treating or preventing a neurodegenerative disorder, such as, for example, acetylcholinesterase inhibitors (i.e. galantamine, donezepil hydrochloride). When used in conjunction with the present invention, that is, in combination therapy, an additive or synergistic effect may be achieved such that many if not all of unwanted side effects can be reduced or eliminated. The reduced side effect profile of these drugs is generally attributed to, for example, the reduced dosage necessary to achieve a therapeutic effect with the administered combination.

The phrase “combination therapy” embraces the administration of a composition of the present invention in conjunction with another pharmaceutical agent that is indicated for treating or preventing a neurodegenerative disorder in a subject, as part of a specific treatment regimen intended to provide a beneficial effect from the co-action of these therapeutic agents for the treatment of a neurodegenerative disorder. The beneficial effect of the combination includes, but is not limited to, pharmacokinetic or pharmacodynamic co-action resulting from the combination of therapeutic agents. Administration of these therapeutic agents in combination typically is carried out over a defined time period (usually substantially simultaneously, minutes, hours, days, weeks, months or years depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules, or tablets for each of the therapeutic agents. Sequential or substantially simultaneous administration of each therapeutic agent can be effected by any appropriate route. The composition of the present invention can be administered orally or nasogastric, while the other therapeutic agent of the combination can be administered by any appropriate route for that particular agent, including, but not limited to, an oral route, a percutaneous route, an intravenous route, an intramuscular route, or by direct absorption through mucous membrane tissues. For example, the composition of the present invention is administered orally or nasogastric and the therapeutic agent of the combination may be administered orally, or percutaneously. The sequence in which the therapeutic agents are administered is not narrowly critical. “Combination therapy” also can embrace the administration of the therapeutic agents as described above in further combination with other biologically active ingredients, such as, but not limited to, an analgesic, for example, and with non-drug therapies, such as, but not limited to, surgery.

The therapeutic compounds which make up the combination therapy may be a combined dosage form or in separate dosage forms intended for substantially simultaneous administration. The therapeutic compounds that make up the combination therapy may also be administered sequentially, with either therapeutic compound being administered by a regimen calling for two step administration. Thus, a regimen may call for sequential administration of the therapeutic compounds with spaced-apart administration of the separate, active agents. The time period between the multiple administration steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by an oral route, a percutaneous route, an intravenous route, an intramuscular route, or by direct absorption through mucous membrane tissues, for example. Whether the therapeutic compounds of the combined therapy are administered orally, by inhalation spray, rectally, topically, buccally, sublingually, or parenterally (for example, subcutaneous, intramuscular, intravenous and intradermal injections), separately or together, each such therapeutic compound will be contained in a suitable pharmaceutical formulation of pharmaceutically-acceptable excipients, diluents or other formulations components.

For oral administration, the pharmaceutical composition can contain a desired amount of a compound of formula (I), and be in the form of, for example, a tablet, a hard or soft capsule, a lozenge, a cachet, a troche, a dispensable powder, granules, a suspension, an elixir, a liquid, or any other form reasonably adapted for oral administration. Illustratively, such a pharmaceutical composition can be made in the form of a discrete dosage unit containing a predetermined amount of the active compound such as a tablet or a capsule. Such oral dosage forms can further comprise, for example, buffering agents. Tablets, pills and the like additionally can be prepared with enteric coatings.

Pharmaceutical compositions suitable for buccal or sublingual administration include, for example, lozenges comprising the active compound in a flavored base, such as sucrose, and acacia or tragacanth, and pastilles comprising the active compound in an inert base such as gelatin and glycerin or sucrose and acacia.

Liquid dosage forms for oral administration can include pharmaceutically acceptable emulsions, solutions, suspensions, syrups, and elixirs containing inert diluents commonly used in the art, such as water. Such compositions can also comprise, for example, wetting agents, emulsifying and suspending agents, and sweetening, flavoring, and perfuming agents.

Examples of suitable liquid dosage forms include, but are not limited, aqueous solutions comprising the active compound and beta-cyclodextrin or a water soluble derivative of beta-cyclodextrin such as sulfobutyl ether beta-cyclodextrin; heptakis-2,6-di-O-methyl-beta-cyclodextrin; hydroxypropyl-beta-cyclodextrin; and dimethyl-beta-cyclodextrin.

The pharmaceutical compositions of the present invention can also be administered by injection (intravenous, intramuscular, subcutaneous). Such injectable compositions can employ, for example, saline, dextrose, or water as a suitable carrier material. The pH value of the composition can be adjusted, if necessary, with suitable acid, base, or buffer. Suitable bulking, dispersing, wetting or suspending agents, including mannitol and polyethylene glycol (such as PEG 400), can also be included in the composition. A suitable parenteral composition can also include an active compound lyophilized in injection vials. Aqueous solutions can be added to dissolve the composition prior to injection.

The pharmaceutical compositions can be administered in the form of a suppository or the like. Such rectal formulations preferably contain the active compound in a total amount of, for example, about 0.075 to about 75% w/w, or about 0.2 to about 40% w/w, or about 0.4 to about 15% w/w. Carrier materials such as cocoa butter, theobroma oil, and other oil and polyethylene glycol suppository bases can be used in such compositions. Other carrier materials such as coatings (for example, hydroxypropyl methylcellulose film coating) and disintegrants (for example, croscarmellose sodium and cross-linked povidone) can also be employed if desired.

The subject compounds may be free or entrapped in microcapsules, in colloidal drug delivery systems such as liposomes, microemulsions, and macroemulsions.

These pharmaceutical compositions can be prepared by any suitable method of pharmaceutics, which includes the step of bringing into association active compound of the present invention and a carrier material or carriers materials. In general, the compositions are uniformly and intimately admixing the active compound with a liquid or finely divided solid carrier, or both, and then, if necessary, shaping the product. For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binding agent, lubricant, inert diluent and/or surface active/dispersing agent(s). Molded tablets can be made by molding, in a suitable machine, the powdered compound moistened with an inert liquid diluent.

Tablets of the present invention can also be coated with a conventional coating material such as Opadry™ White YS-1-18027A (or another color) and the weight fraction of the coating can be about 3% of the total weight of the coated tablet. The compositions of the present invention can be formulated so as to provide quick, sustained or delayed release of the compositions after administration to the patient by employing procedures known in the art.

When the excipient serves as a diluent, it can be a solid, semi-solid or liquid material, which acts as a vehicle, carrier or medium for the active ingredient. Thus, the compositions can be in the form of tablets, chewable tablets, pills, powders, lozenges, sachets, cachets, elixirs, suspensions, emulsions, solutions, syrups, aerosols (as a solid or in a liquid medium), soft and hard gelatin capsules and sterile packaged powders.

In one embodiment of the present invention, the manufacturing processes may employ one or a combination of methods including: (1) dry mixing, (2) direct compression, (3) milling, (4) dry or non-aqueous granulation, (5) wet granulation, or (6) fusion. Lachman et al., The Theory and Practice of Industrial Pharmacy (1986).

In another embodiment of the present invention, solid compositions, such as tablets, are prepared by mixing a therapeutic agent of the present invention with a pharmaceutical excipient to form a solid preformulation composition containing a homogeneous mixture of the therapeutic agent and the excipient. When referring to these preformulation compositions(s) as homogeneous, it is meant that the therapeutic agent is dispersed evenly throughout the composition so that the composition may be readily subdivided into equally effective unit dosage forms, such as tablets, pills and capsules. This solid preformulation is then subdivided into unit dosage forms of the type described herein.

Compressed tablets are solid dosage forms prepared by compacting a formulation containing an active ingredient and excipients selected to aid the processing and improve the properties of the product. The term “compressed tablet” generally refers to a plain, uncoated tablet for oral ingestion, prepared by a single compression or by pre-compaction tapping followed by a final compression.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. A variety of materials can be used for such enteric layers or coatings, including a number of polymeric acids and mixtures of polymeric acids with such materials as shellac, cetyl alcohol and cellulose acetate.

Use of a long-term sustained release implant may be suitable for treatment of neurodegenerative disorders in patients who need continuous administration of the compositions of the present invention. “Long-term” release, as used herein, means that the implant is constructed and arranged to deliver therapeutic levels of the active ingredients for at least 30 days, and preferably 60 days. Long-term sustained release implants are well known to those of ordinary skill in the art and include some of the release systems described above.

In another embodiment of the present invention, the compound for treating a neurodegenerative disorder comes in the form of a kit or package containing one or more of the therapeutic compounds of the present invention. These therapeutic compounds of the present invention can be packaged in the form of a kit or package in which hourly, daily, weekly, or monthly (or other periodic) dosages are arranged for proper sequential or simultaneous administration. The present invention further provides a kit or package containing a plurality of dosage units, adapted for successive daily administration, each dosage unit comprising at least one of the therapeutic compounds of the present invention. This drug delivery system can be used to facilitate administering any of the various embodiments of the therapeutic compounds of the present invention. In one embodiment, the system contains a plurality of dosages to be administered daily or weekly. The kit or package can also contain the agents utilized in combination therapy to facilitate proper administration of the dosage forms. The kits or packages also contain a set of instructions for the subject.

It is believed that one skilled in the art, based on the description herein, can utilize the present invention to its fullest extent. The following specific examples are therefore to be construed as merely illustrative, and not limitative of the remainder of the disclosure in any way whatsoever.

### EXAMPLE I

Materials

Aβ₁₋₄₂ and Aβ peptide fragments were purchased from American Peptide Co. (Sunnyvale, Calif.). Polyclonal rabbit anti-β-amyloid peptide (cat. no. 71-5800) was obtained from Zymed Laboratories (San Francisco, Calif.). [22-³H]R-hydroxycholesterol (sp. act. 20 Ci/mmol) was synthesized by American Radiolabeled Chemical (St Louis, Mo.). Cholesterol, 22R-hydroxycholesterol, 22S-hydroxycholesterol, pregnenolone, 17α-hydroxypregnenolone and DHEA were purchased from Sigma-Aldrich (St. Louis, Mo.). Cell culture supplies were purchased from GIBCO (Grand Island, N.Y.), and cell culture plasticware was from Corning (Corning, N.Y.). Electrophoresis reagents and materials were supplied from Bio-Rad (Richmond, Calif.). All other chemicals used were of analytical grade and were obtained from various commercial sources.

Tissue Samples

All human tissue samples were obtained from the Harvard Brain Tissue Resource Center (Belmont, Mass.). Samples for steroid measurements were either snap frozen or passively frozen in liquid nitrogen. Brain hippocampus and frontal cortex samples were obtained from 19 patients, 12 AD (6 men and 6 women) and 7 age-matched control patients (4 men and 3 women). AD patients were classified by the Harvard Tissue Resource Center as having “severe AD.” Mean age for all patients was 74.6±7.2 years for AD patients and 73.4±10.5 years for control. Mean post-mortem interval was 10.2 hours for AD patients and 14.7 hours for control. Protocols for the use of human tissue were approved by the Georgetown University Internal Review Board.

Purification and Measurement of 22R-hydroxycholesterol

Samples were extracted and purified by reverse phase HPLC as previously described. Brown, R. C., Cascio, C. & Papadopoulos, V. (2000) J. Neurochem. 74, 847-859. Fractions containing 22R-hydroxycholesterol were collected (retention time of 22R-hydroxycholesterol=55 minutes) and levels of 22R-hydroxycholesterol were determined using the cholesterol oxidase assay. Gamble, W., Vaughan, M., Kruth, H. S. & Avigan, J. (1978) J. Lipid Res. 19, 1068-1070.

Cell Culture, Cellular Toxicity & Viability Assays

Rat PC12 cells were cultured as previously described. Yao, Z., Drieu K. & Papadopoulos, V. (2001) Brain Res. 889, 181-190. Human NT2 precursor (Ntera2/D1 teratocarcinoma) cells were obtained from Stratagene (La Jolla, Calif.) and cultured following the instructions of the supplier. Differentiated human NT2 neurons (NT2N) were obtained after treatment of the NT2 precursor cells with retinoic acid. Andrews, P. W. (1984) Dev. Biol. 103, 285-293. Aβ was dissolved in media and used either in the aggregated (left overnight at 4° C.) or soluble (containing oligomers such as dimers and tetramers) forms examined by electrophoresis as previously described. Yao, Z. et al., Brain Res. (2001). Cellular toxicity for Aβ and Aβ fragments was assayed using the 3-(4,5-dimethylthiazol-2-yl)-2,5-diphenyl tetrazolium bromide (MTT) assay (Trevigen, Gaithersburg, Md.) as previously described. Id. Cell viability was measured using the trypan blue exclusion method as previously described. Id. In brief, for these studies, cells were treated for 72 h with steroids in the presence or absence of increasing concentrations of Aβ. At the end of the incubation, the cells were washed three times with PBS and incubated for 15 min with 0.1% trypan blue stain solution at room temperature. After washing three times with PBS, 0.1 N NaOH was added to the cells and trypan blue staining was quantified using the Victor quantitative detection spectrophotometer (EGG-Wallac, Gaithersburg, Md.) at 450 nm. Cell protein levels were determined in the same samples by the method of Bradford (Bradford, M. M. (1976) Anal. Biochem. 72, 248-254), where coomassie blue staining is detected at 590 nm.

Cholesterol-Protein Binding Blot Assay (CPBBA)

Purified Aβ₁₋₄₂ protein (50 μM) or various Aβ fragments (50 μM) and ³H-22R-hydroxycholesterol were incubated either alone or in the presence of increasing concentrations of unlabeled 22R-hydroxycholesterol in 20 μl volume for 24 h at 37° C. At the end of the incubation time, samples were separated by 1.5% agarose (Type I-B) gel electrophoresis and transferred to nitrocellulose membrane (Schleicher & Schuell, Keene, N.H.) in 10×SSC buffer. The membrane was exposed to tritium-sensitive screen and analyzed by phosphoimaging using the Cyclone Storage phosphor system (Packard BioScience, Meridien, Conn.). Image-densitometric analysis was performed using the OptiQuant software (Packard). This method allows for the separation, visualization and identification of Aβ complexes, which have incorporated radiolabeled cholesterol (Yao, Z. & Papadopoulos, V., manuscript submitted) and 22R-hydroxycholesterol under native conditions. Low molecular weight unincorporated 22R-hydroxycholesterol is separated and eliminated during electrophoresis.

Aβ Aggregation Assay

Purified Aβ₁₋₄₂ protein (50 mM) in cell culture media was incubated either alone or in the presence of increasing concentrations of 22R-hydroxycholesterol for 24 h at 37° C. At the end of the incubation, proteins were separated by SDS-PAGE on 4-20% gradient acrylamide-bis-acrylamide gel at 125V for 2 h. Proteins were visualized by coomassie blue staining. Aβ species were identified by immunoblot analysis. Yao, Z. et al., Brain Res. (2001).

Immunoblot Analysis

The membrane with the 22R-hydroxycholesterol-Aβ peptide complexes was then used to examine Aβ levels. Membranes were blocked by incubating the nitrocellulose in 5% milk and treated for immunodetection of Aβ using ECL reagents (Amersham-Pharmacia, Piscataway, N.J.). Li, H., Yao, Z., Degenhardt, B., Teper, G. & Papadopoulos, V. (2001) Proc. Natl. Acad. Sci. USA 98, 1267-1272. Anti-Aβ antibody and secondary antibodies were used at 0.2 μg/ml and 1:5000 dilution, respectively.

Peptide Modeling and 22R-hydroxycholesterol Docking

Computer docking of 22R-hydroxycholesterol with Aβ₁₇₋₄₀ and Aβ₂₅₋₃₅ was accomplished using a Aβ structure generated from the solution structure of Aβ₁₋₄₀OMet(O) (MMDB Id: 7993 PDB Id: 1BA) resulting from data generated by CD and NMR spectroscopy. Watson, A. A., Fairlie, D. P., & Craik, D. J. (1998) Biochemistry 37, 12700. The Met(O) SME 35 residue was replaced by Met retaining the adjacent backbone dihedral angles and the coordinates for residues 17-40 extracted. The 22R-hydroxycholesterol structure was developed using the Alchemy 2000 program (Tripos, St. Louis, Mo.). The docking was accomplished using Monte Carlo simulated annealing (Li, H. et al., Proc. Natl. Acad. Sci. USA (2001)) and implemented in modified versions of Autogrid/Autodock. Morris, G. M., Goodsell, D. S., Halliday, R. S., Huey, R., Hart, W. E., Belew, R. K., & Olson, A. J.(1998). J. Comput. Chem. 19, 1639-1662. The conformation of minimum energy of approximately 10⁹ conformations was evaluated. Five sessions consisting of 100 runs, each starting at a random initial relative location and orientation of the ligand with the target were executed. Each run was comprised of 100 annealing cycles using about 2×10⁴ improvement steps. The total computation time using the modified program was about 15 minutes using a 1.7 GHz, 1 GB RAM PC.

Statistics

Statistical analysis was performed by one-way analysis of variance (ANOVA) and unpaired Student's t test using the INSTAT 3.00 package (GraphPad, San Diego, Calif.).

Results

As depicted in FIG. 2, endogenous 22R-hydroxycholesterol levels in human brain were measured by the cholesterol oxidase assay after HPLC purification. Data presented is means±SEM for duplicate measurements from 12 AD and 7 age-matched control samples. FIG. 2 shows that levels of 22R-hydroxycholesterol in hippocampus of AD patient's brain specimens were decreased by 60% (p=0.04) compared to age-matched controls. 22R-hydroxycholesterol levels were also decreased by 50% in frontal cortex of AD patient's brain specimens compared to age-matched controls, although in a non-significant manner.

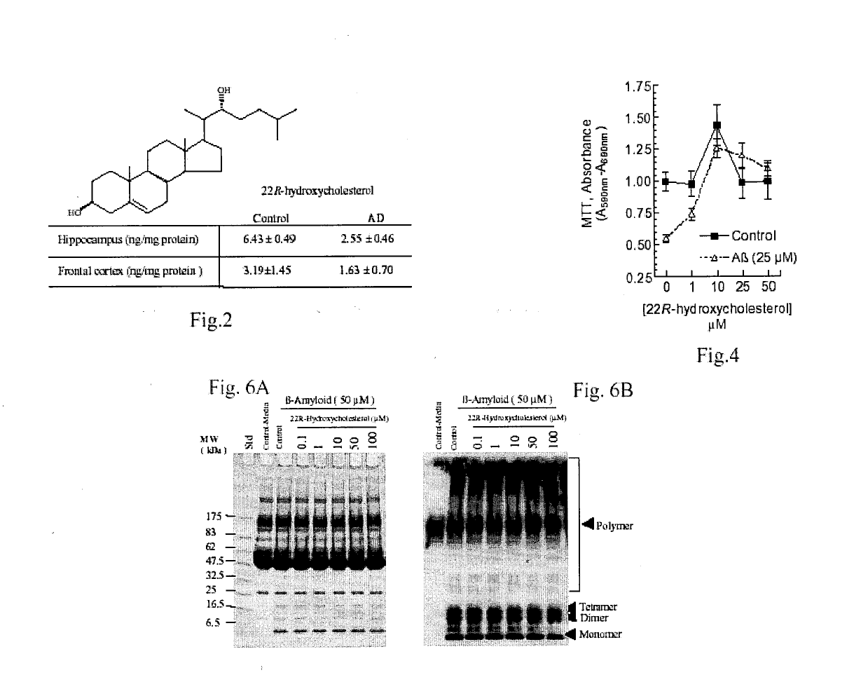

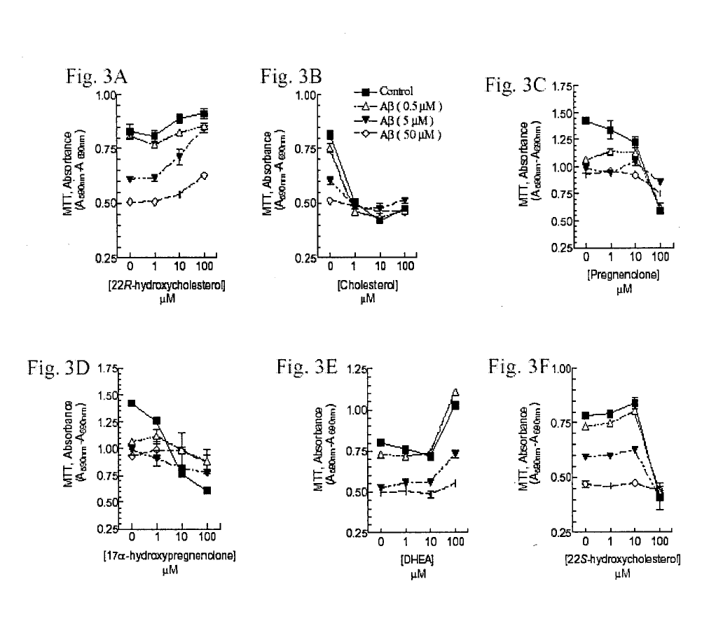

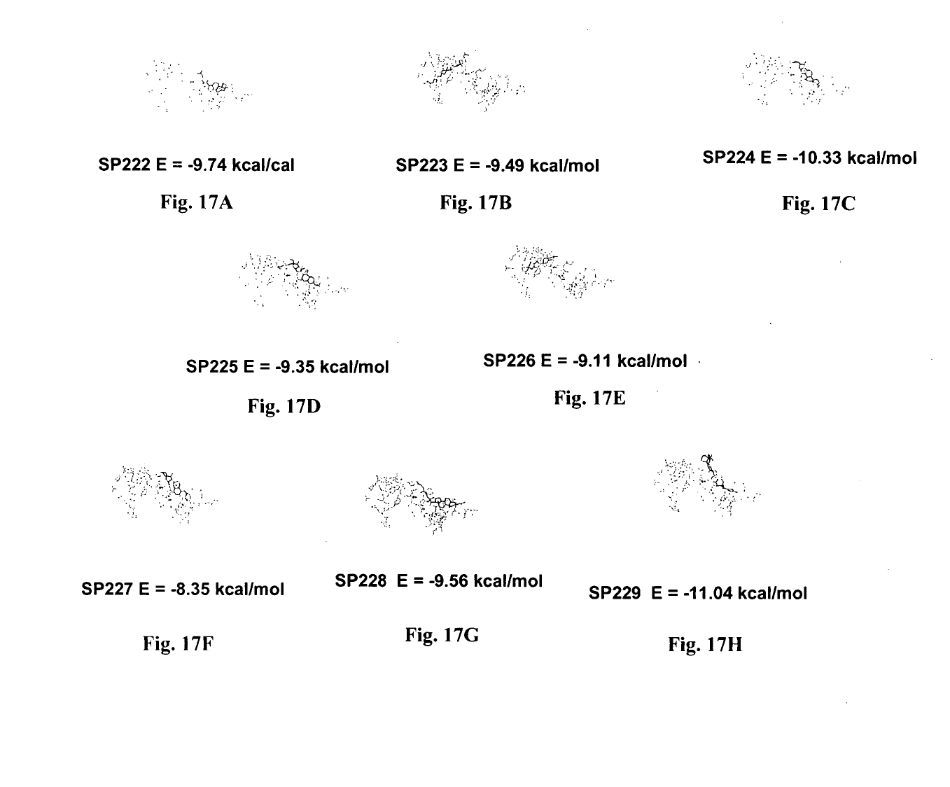

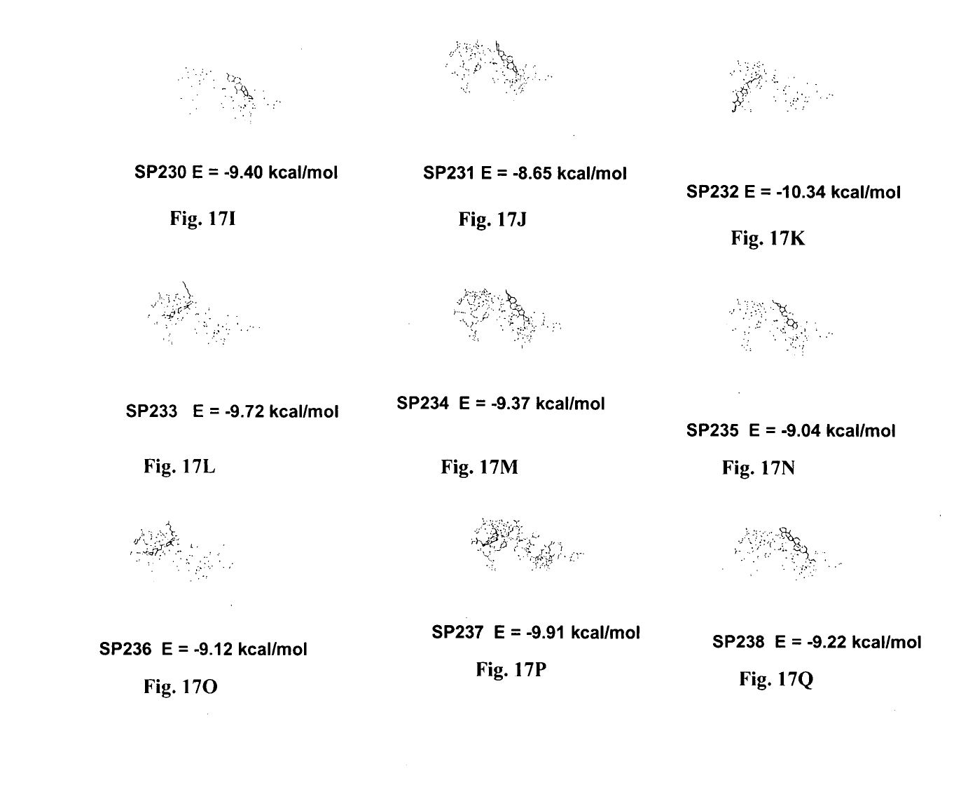

PC12 cells were treated for 24 h with the indicated concentrations of Aβ₁₋₄₂ in the absence or presence of increasing concentrations of 22R-hydroxycholesterol (FIG. 3A), cholesterol (FIG. 3B), pregnenolone (FIG. 3C) or 17α-hydroxypregnenolone (FIG. 3D), DHEA (FIG. 3E) or 22S-hydroxycholesterol (FIG. 3F). Results shown are means±SD (n=6-12). The ability of 22R-hydroxycholesterol to rescue rat PC12 neuronal cells from Aβ-induced cytotoxicity was examined using the mitochondrial diaphorase assay MTT.

Aβ₁₋₄₂ induced a dose-dependent neurotoxicity that reached 26% (p<0.001) and 40% (p<0.001) cell death in the presence of 5.0 and 50 μM Aβ, respectively (FIG. 3A). Increasing concentrations of 22R-hydroxycholesterol did not affect PC12 cell viability, although a non-significant improvement was seen in the presence of 10 and 100 μM of 22R-hydroxycholesterol (FIG. 3A). 22R-hydroxycholesterol was able to rescue all the cells from 25 μM Aβ-induced cytotoxicity (p<0.001) and to rescue 50% (p<0.01) of the cells dying in the presence of 50 μM Aβ (FIG. 3A). Interestingly, 22R-hydroxycholesterol was effective only when present at the same time with Aβ. Pretreatment of PC12 cells with 22R-hydroxycholesterol followed by treatment with Aβ failed to offer any protection to the cells (data not shown).

The neuroprotective effect of 22R-hydroxycholesterol could not be replicated using either its precursor cholesterol (FIG. 3B) or its metabolite pregnenolone (FIG. 3C). In contrast, both cholesterol and pregnenolone alone were toxic to the cells. Moreover, the presence of cholesterol accentuated the toxic effect of low concentrations of Aβ. 17α-hydroxypregnenolone alone was also toxic to the cells (FIG. 3D). 100 μM DHEA had a positive effect on cell viability. The same concentration of DHEA protected against the 5 μM (p<0.001), but not 50 μM, Aβ-induced cytotoxicity (FIG. 3E). The effect of 22R-hydroxycholesterol was stereospecific because 22S-hydroxycholesterol not only failed to protect against the Aβ-induced neurotoxicity, but at a 100 μM concentration was neurotoxic (FIG. 3F).

It should be noted that, in the presented studies, aggregated Aβ (left overnight at 4° C.) was used. In separate experiments, soluble Aβ (containing oligomers) was directly added to PC12 cells and found to be toxic (data not shown). 22R-hydroxycholesterol also protected against the Aβ oligomer-induced toxicity (not shown).

The neuroprotective effect of 22R-hydroxycholesterol was not restricted to PC12 cells but was replicated on differentiated human NT2N neurons (FIG. 4). Differentiated human NT2N neurons were treated for 72 h with 25 μM Aβ₁₋₄₂ in the presence or absence of 22R-hydroxycholesteol. 25 μM Aβ inhibit by 50% (p<0.001) human neuron viability, while 1 and 10 μM 22R-hydroxycholesterol protected by 50% (p<0.01) and 100% (p<0.001), respectively, against the Aβ-induced toxicity (FIG. 4). To assess whether 22R-hydroxycholesterol rescues human NT2 cells against other toxic insults, NT2 cells were treated for three days with 5 mM glutamate in the presence or absence of 1 to 50 mM 22R-hydroxycholesterol. Glutamate induced a 30% decrease in cell viability, determined using the MTT assay and the presence of 22R-hydroxycholesterol failed to protect the cells (data not shown).

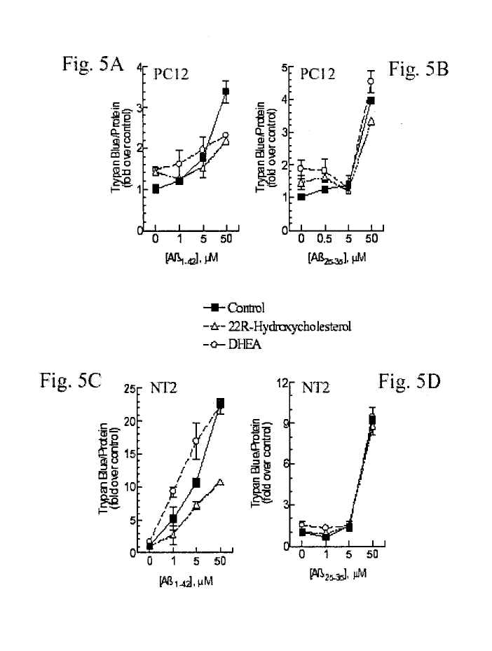

The results obtained from using MTT assay were further confirmed with the trypan blue dye exclusion assay. PC12 cells were treated for 72 h with increasing concentrations of Aβ₁₋₄₂ (FIG. 5A) or Aβ₂₅₋₃₅ (FIG. 5B) in the presence or absence of 100 μM 22R-hydroxycholesterol or DHEA. NT2 cells were treated for 72 h with increasing concentrations of Aβ₁₋₄₂ (FIG. 5C) or Aβ₂₅₋₃₅ (FIG. 5D) in the presence or absence of 25 μM 22R-hydroxycholesterol or DHEA. Levels of viability were measured using the trypan blue assay as described under Materials and Methods. Results are expressed as fold trypan blue stained cells per total cell protein over control untreated cells. Results shown are means±SD (n=6-12). FIGS. 5A and 5C show that 22R-hydroxycholesterol rescued both the rat PC12 (FIG. 5A) and human NT2 (FIG. 5C) cells from Aβ₁₋₄₂-induced cell death. In contrast, DHEA only protected the rat PC12 cells from Aβ₁₋₄₂-induced cell death but not NT2 cells (FIGS. 5A and 5C). Neither 22R-hydroxycholesterol nor DHEA could rescue the PC12 and NT2 cells from the Aβ₂₅₋₃₅-induced cell death (FIGS. 5B and 5D).

The ability of 22R-hydroxycholesterol to alter Aβ aggregation was also examined. Purified Aβ₁₋₄₂ protein (50 μM) in cell culture media was incubated either alone or in the presence of increasing concentrations of the 22R-hydroxycholesterol for 24 h at 37° C. At the end of the incubation proteins were separated by SDS-PAGE and visualized by coomassie blue (FIG. 6A). Aβ species formed were identified by immunoblotting using an anti-Aβ polyclonal antiserum (FIG. 6B). Aβ aggregation can be seen on the top of the gel and it is absent in control-media lane. FIGS. 6A and 6B show that 22R-hydroxycholesterol did not affect Aβ aggregation identified by immunoblot analysis (FIG. 6B) of the coomassie blue stained gels (FIG. 6A). A 100 kDa band recognized by the Aβ polyclonal antiserum used in all samples, including control-media, probably reflects non-specific binding of the antiserum.

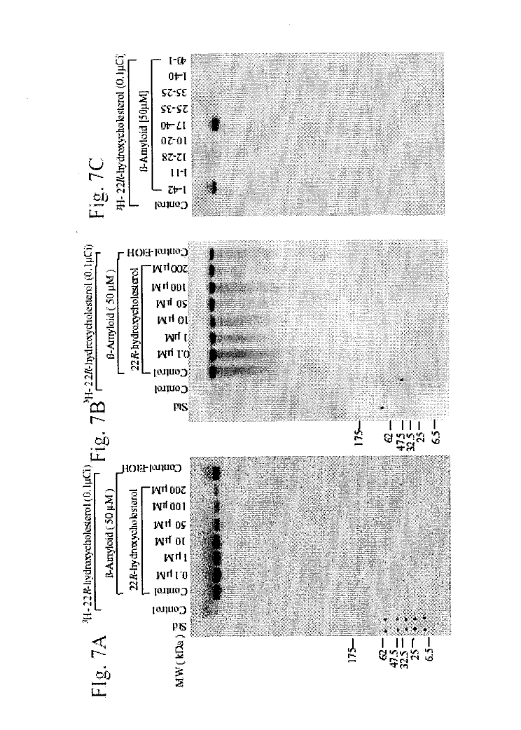

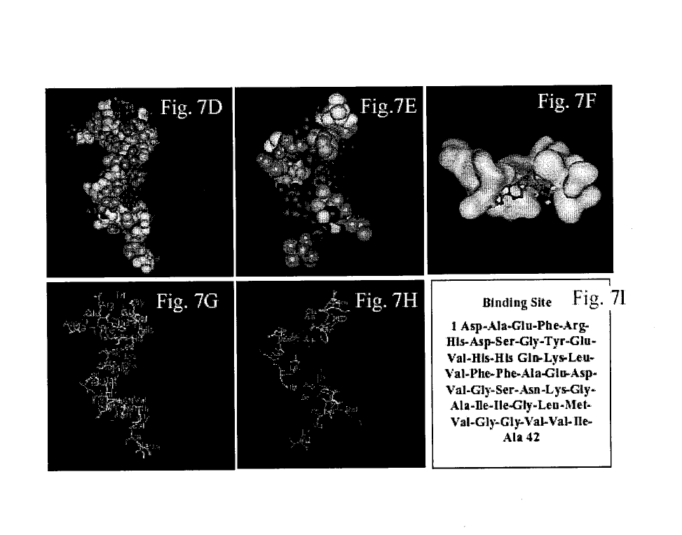

The mechanism of action of 22R-hydroxycholesterol was then examined. Considering that 22R-hydroxycholesterol was neuroprotective only when in presence of Aβ, the direct interaction between 22R-hydroxycholesterol and Aβ was explored with a novel method, the CPBBA method. Co-incubation of radiolabeled 22R-hydroxycholesterol together with Aβ₁₋₄₂ for 24 hours at 37° C. demonstrated the presence of a high molecular weight radiolabeled band (FIG. 7A) recognized by an antibody specific to Aβ (FIG. 7B). The specificity of the radiolabeling of Aβ₁₋₄₂ by 22R-hydroxycholesterol was demonstrated by competition studies using unlabeled 22R-hydroxycholesterol (FIG. 7A). In these studies, 50 and 200 μM 22R-hydroxycholesterol inhibited by 50 and 90%, respectively, the binding of radiolabeled 22R-hydroxycholesterol to 50 μM Aβ₁₋₄₂, as indicated by image analysis of the radiolabeled Aβ₁₋₄₂ (FIG. 7A). Equal loading of Aβ₁₋₄₂ in the incubation reactions and in CPBBA was assessed by immunoblot analysis of the radiolabeled Aβ₁₋₄₂ (FIG. 7B). It should be noted that, despite the decreased radiolabeling of Aβ₁₋₄₂ observed in the presence of 50-200 μM 22R-hydroxycholesterol, there were no differences in the amount of Aβ₁₋₄₂ present in each lane. These data demonstrate that, under native conditions, 22R-hydroxycholesterol binds to Aβ. Using CPBBA and various Aβ synthetic peptides, the 22R-hydroxycholesterol-binding site in Aβ was mapped to amino acids 17-40 of Aβ (FIGS. 7C and 7E). Interestingly peptide Aβ₂₅₋₃₅, which maintained its neurotoxicity in the presence of 22R-hydroxycholesterol (FIGS. 7B and 7D), did not bind 22R-hydroxycholesterol (FIG. 7C). These data were further confirmed using computational docking simulations. The docking results show that Aβ₁₇₋₄₀ forms a pocket where 22R-hydroxycholesterol could dock (FIG. 7D). The pocket formed by amino acids G₂₉A₃₀I₃₁ captures the C₂₇₋₂₉ atoms of 22R-hydroxycholesterol. The orientation R, versus S, is permissive for 22R-hydroxycholesterol docking. Similar studies using Aβ325-35 indicated that, despite the presence of some of the amino acids present in the 19-36 area, the docking energy of Aβ₂₅₋₃₅ for 22R-hydroxycholesterol (−6.0510 kcal/mol) is high relative to Aβ₁₇₋₄₀ (−8.6939 kcal/mol) and to Aβ₁₋₄₂ (−9.6960 kcal/mol), suggesting that this steroid does not bind to Aβ₂₅₋₃₅ in agreement with the CPBBA data.

Discussion

In the brain, neurosteroids (pregnenolone and DHEA) accumulate independently of the supply by peripheral endocrine organs (Baulieu, E. E.& Robel, P. (1990) J. Steroid Biochem. Mol. Biol. 37,395-403), act as neuromodulators (Paul, M. P. & Purdy, R. H. (1992) FASEB J. 6, 2311-2322) and might serve as pharmacological tools for various neuropathologies (Costa, E., Cheney, D. L., Grayson, D. R., Korneyev, A., Longone, P., Pani, L., Romeo, E., Zivkovich, E. & Guidotti, A. (1994) Ann. N.Y. Acad. Sci. 746, 223-242). Glial cells can convert cholesterol to pregnenolone. In vitro studies show that oligodendrocytes, a glioma cell line and Schwann cells express the cytochrome P450 responsible for the side chain cleavage of cholesterol and thus pregnenolone formation. Jung-Testas, I., Hu, Z., Baulieu, E. E. & Robel, P. (1989) Endocrinology 125, 2083-2091; Papadopoulos, V., Guarneri, P., Krueger, K. E., Guidotti, A. & Costa, E.(1992) Proc. Natl. Acad. Sci. USA 89, 5113-5117; Akwa, Y., Schumacher, M., Jung-Testas, I. & Baulieu, E. E (1993) C. R. Acad. Sci III (France) 316, 410-414. The P450 side chain cleavage enzyme is also present in rodent brain (Stromstedt, M. & Waterman, M. R. (1995) Mol. Brain Res. 34, 75-88) and in both AD and age-matched control human brain specimens (Brown, R. C., Han, J. Cascio, C. & Papadopoulos, V. (2002) Neurobiol. Aging, in press). It has been well described that during this enzymatic reaction one of the three hydroxylated intermediates formed is 22R-hydroxycholesterol. Dixon, R., et al., Biochem. Biophys. Res. Commun. (1970); Hall, P. F. (1985) Vitamins & Hormones 42, 315-368. 22R-hydroxycholesterol is more polar than cholesterol and is easily transported through cell membranes. In the present study, the levels of 22R-hydroxycholesterol were found to be lower in AD patient's brain specimens compared to age-matched controls. Levels of 22R-hydroxycholesterol were significantly decreased in hippocampus, a structure in the limbic system of the brain that is critical to cognitive functions, as learning and memory, and is affected in AD. The physiological function of Aβ is to control cholesterol transport (Yao, Z. & Papadopoulos, V., FASEB Journal, 16:1677-1679). Based on this finding, the decrease of 22R-hydroxycholesterol might be due to the overproduction of Aβ in AD patient's brain (Roher, A. E., Lowenson, J. D., Clark, S., Wolkow, C., Wang, R., Cotter, R. J., Reardon, I. M., Zurcher-Neely, H. A., Heinrikson, R. L., Ball, M. J.& Greenberg, B. D. (1993) J. Biol. Chem. 286, 3072-3083; Younkin, S. G. (1998) J. Physiol. 92,289-292) that blocks cholesterol trafficking or decreases cholesterol uptake by the cells, thus affecting the availability of the substrate cholesterol for neurosteroid formation resulting in decreased synthesis of 22R-hydroxycholesterol in AD patient's brain. Alternatively, increased de novo synthesis of pregnenolone and DHEA from cholesterol in AD brain specimens will also exhaust the available intermediate 22R-hydroxycholesterol in AD. The presence of increased levels of pregnenolone and DHEA in AD hippocampus (Brown, R. C., Han, Z., Cascio, C. & Papadopoulos, V. (2003) Neurobiology of Aging, 24:57-65)), is induced by Aβ (Brown, R. C., Cascio, C. & Papadopoulos, V. (2000) J. Neurochem. 74:847-859). It is also possible that both events, Aβ-induced decrease in cholesterol trafficking and increase in cholesterol metabolism might occur in AD and lead to decreased 22R-hydroxycholesterol levels.

For these studies, a well-established rat PC12 neuronal cell model was used. However, the neuroprotective effect of 22R-hydroxycholesterol was not restricted to rodent neurons but it was also seen in human NT2 and NT2N neuronal cells. NT2 cells is a clonal line of human teratocarcinoma cells and NT2N, derived from NT2 cells, are post-mitotic, terminally differentiated neurons that possess cell surface markers consistent with neurons of the central nervous system. Andrews, P. W., Dev. Biol. (1984). 22R-hydroxycholesterol was found to protect both rat and human neurons from Aβ-induced toxicity in a dose-dependent manner with IC₅₀s of 10 and 3 μM for PC12 and NT2T cells, respectively. Treatment of the cells with 22R-hydroxycholesterol offered full protection against Aβ used at 25 μM concentration and 50% neuroprotection against the peptide used at 50 μM.

A number of steroids have been tested for their putative neuroprotective properties against Aβ, examined using the amyloid fibril-induced MTT formazan exocytosis assay in B12 rat neural cells. Liu Y. & Schubert, D. (1998) J. Neurochem. 71, 2322-2329. In these studies, Liu and Schubert suggested that compounds that block amyloid fibril-induced MTT formazan exocytosis, without affecting that of control cells, should be acting at upstream targets and thus be neuroprotective. Id. Using the MTT assay, which measures the formation of blue formazan, in addition to the effect of 22R-hydroxycholesterol, the neuroprotective properties of various steroids involved in the metabolism of cholesterol was examined. From the steroids tested on Aβ-induced PC12 neurotoxicity, all were toxic except for 22R-hydroxycholesterol and DHEA. The neuroprotective effect of DHEA on rodent neurons is in agreement with previous studies. Kimonides, V G, Khatibi, N H, Svendsen, C N, Sofroniew, M V, & Hervert, J (1998) Proc. Natl. Acad. Sci. USA, 95, 1852-1857; Cardounel, A, Regelson, W, & Kalimi, M (2000) Proc. Soc. Exp. Biol. Med., 222, 145-149. However, in contrast to 22R-hydroxycholesterol, DHEA had no effect on Aβ-induced human NT2 cell death, suggesting that the effect of 22R-hydroxycholesterol is not species specific, probably because this steroid interacts directly with Aβ. The precursor of 22R-hydroxycholesterol, cholesterol, was found to be neurotoxic. However, the presence of an hydroxyl group at carbon 22(R) not only relieves the toxic effect of cholesterol but also protects against Aβ-induced neurotoxicity. The specificity of the effect of 22R-hydroxycholesterol is further evidenced by the observation that its enantiomer 22S-hydroxycholesterol is inactive and at high concentrations neurotoxic.

The direct interaction between 22R-hydroxycholesterol and Aβ was shown using a novel assay, the CPBBA. This assay allows for the study and visualization of the direct interaction, under native conditions, between the radiolabeled steroid and Aβ, or Aβ peptide fragments. Radiolabeled 22R-hydroxycholesterol binds Aβ and the unlabeled 22R-hydroxycholesterol displaces the bound steroid. CPBBA indicated that 22R-hydroxycholesterol binds to Aβ₁₋₄₂ and Aβ₁₇₋₄₀, but barely interacts with Aβ₁₋₄₀. Mass spectrometric analysis of purified amyloid plaques revealed that Aβ₁₋₄₂ is the principal component of amyloid deposits, therefore, Aβ₁₋₄₂ is believed to be the main culprit in the pathogenesis of AD. Roher, A. E., et al., J. Biol. Chem. (1993); Younkin, S. G., J. Physiol. (1998). The shorter Aβ form of 40 amino acids is believed to have no pathologic effect (Brown, R. C., et al., J. Neurochem. (2000)) and is less abundant in AD brain (Roher, A. E., et al., J. Biol. Chem. (1993); Younkin, S. G., J. Physiol. (1998)). Computational modeling simulations based on the reported structure of Aβ indicated that amino acids 19-36 capture capture the side chain of 22-Rhydroxycholesterol when the hydroxyl group has the R orientation. Interestingly, the peptide Aβ₂₅₋₃₅ that is known for its toxic effects (Schubert, D., Behl, C., Lesley, R., Brack, A., Dargusch, R., Sagara, Y. & Kimura H. (1995) Proc. Natl. Acad. Sci. USA 92, 1989-1993) retained its neurotoxic property even in presence of 22R-hydroxycholesterol. Computational modeling simulations and CPBBA failed to show an interaction between 22R-hydroxycholesterol and peptide Aβ₂₅₋₃₅, suggesting that it is the three dimensional conformation of Aβ₁₋₄₂ and Aβ₁₇₋₄₀ that confers the ability of amino acids 19-36 to interact with 22R-hydroxycholesterol rather than the primary amino acid sequence.

22R-hydroxycholesterol binding to amino acids 17-40 of Aβ₁₋₄₂ leads to the protection/rescuing of both rodent and human neuronal cells from the Aβ₁₋₄₂-induced cytotoxicity and cell death. The exact mechanism by which 22R-hydroxycholesterol acts to block the neurotoxic effect of Aβ is not known. However, the data presented herein indicated that it does not affect Aβ polymerization. Binding of 22R-hydroxycholesterol to Aβ₁₋₄₂ might either change the conformation of the Aβ monomer or polymer, thus rendering it inactive, or prohibit Aβ from interacting with the cell or activating intracellular mechanism mediating its toxic effect. Thus, the low levels of 22R-hydroxycholesterol in AD patient's brain compared to age-matched controls, in addition to the increased production of Aβ₁₋₄₂ in AD brains, results in decreased/lost ability of the brain to fight against the Aβ₁₋₄₂-induced neurotoxicity. This might be particularly true for presenilin 1-liked familial Alzheimer's disease (FAD) patients, who have the highest levels of Aβ₁₋₄₂. Borchelt, D. R., Thinakaran, G., Eckman, C. B., Lee, M. K., Davenport F., Ratovitsky, T., Prada, C-M., Kim, G., Seekins, S., Yager, D., Slunt, H. H., Wang, R., Seeger M., Levey A. I., Gandy S. E., Copeland N. G., Jenkins N. A., Price D L, Younkin S. G. & Sisodia S. S. (1996), Neuron 17, 1005-1013.

### EXAMPLE II

Materials

Aβ₁₋₄₂ peptide was purchased from American Peptide Co. (Sunnyvale, Calif.). 22R-hydroxycholesterol (SP222) was purchased from Sigma (St Louis, Mo.). [22-3H]R-hydroxycholesterol (sp. act. 20 Ci/mmol) was synthesized by American Radiolabeled Chemical (St Louis, Mo.). The 22R-hydroxycholesterol derivatives (SP223-238) were purchased from Interbioscreen (Moscow, Russia). Cells culture supplies were purchased form GIBCO (Grand Island, N.Y.) and cell culture plasticware was from Corning (Corning, N.Y.) and Packard BioSciences Co. (Meriden, Conn.).

In Silico Screening for 22R-hydroxycholesterol Derivatives

The Interbioscreen Database of naturally occurring entities was screened for compounds containing the 22R-hydroxycholesterol structure using the ISIS software (Information Systems, Inc., San Leandro, Calif.). The structure of the selected and tested 22R-hydroxycholesterol (SP222) and derivatives (SP223-238) are shown in FIG. 1 and the denomination, chemical name and origin for each of these compounds is shown in Table 1.

Cell Culture and Treatments

PC12 cells (rat pheochromocytoma neurons) from ATCC (Manassas, Va.) were cultured at 37° C. and 5% CO2 in RPMI 1640 medium devoid of glutamine and supplemented with 10% fetal bovine serum and 5% horse serum. Yao Z, Drieu K and Papadopoulos V., The Gingko biloba extract EGb 761 rescues PC12 neuronal cells from β-amyloid-induced cell death by inhibiting the formation of β-amyloid-derived diffusible neurotoxic ligands, Brain Res 2001, 889:181-190. Cells were seeded in 96-well plates (8×104 cells/well). After an overnight period of incubation, increasing concentrations of aggregated Aβ (0.1, 1 and 10 μM) were added to the cells in the presence or absence of the indicated concentrations of the SP compounds to be tested. After 72-hours incubation time various parameters, markers of cell viability, were determined. Mouse MA-10 tumor Leydig cells were maintained at 37° C. in DMEM/Ham's F12 (Biofluids, Rockville, Md.) medium supplemented with 5% heat-inactivated fetal calf serum and 2.5% horse serum in 5% CO₂. Cells were plated on 96-well plates at the density of 2.5×104 cells/well for overnight. The cells were stimulated with the indicated concentrations of the various SP compounds in 0.2 ml/well serum-free medium for 2 h. The culture medium was collected and tested for progesterone production by radioimmunoassay.

MTT Cytotoxicity Assay

The cellular toxicity of Aβ was assessed using the 3-(4,5-dimethylthiazol-2-yl)-2,5-diphenyl tetrazolium bromide (MTT) assay (Trevigen, Gaithersburg, Md.). Briefly, 10 μl of the MTT solution were added to the cells cultured in 100 μl medium. After an incubation period of 4 hours, 100 μl of detergent were added and cells were incubated overnight at 37° C. Formazan blue formation was quantified at 600 nm and 690 nm using the Victor quantitative detection spectrophotometer (EGG-Wallac, Gaithersburg, Md.) and the results expressed as (DO600-DO690). Although the MTT assay has been widely used to assess cytotoxicity in neuronal cells treated with Aβ it has been suggested that the results obtained in the presence of various steroids might reflect the Aβ-dependent vesicle recycling leading to increased MTT formazan exocytosis and loss. Liu Y and Schubert D, Steroid hormones block amyloid fibril-induced 3-(4,5-dimethylthiazol-2-yl)-2,5-diphenyltetrazolium bromide (MTT) formazan exocytosis: relationship to neurotoxicity, J Neurochem 1998, 19: 1639-1662. For that reason, additional cytotoxicity and cell viability assays were used.

Trypan Blue Cell Viability Measurement

Cell viability was measured using the trypan blue exclusion method as we previously described. Yao Z, Drieu K and Papadopoulos V, The Gingko biloba extract EGb 761 rescues PC12 neuronal cells from β-amyloid-induced cell death by inhibiting the formation of β-amyloid-derived diffusible neurotoxic ligands, Brain Res 2001, 889:181-190. In brief, cells were treated for 72 h with SP compounds in the presence or absence of increasing concentrations of Aβ. At the end of the incubation, cells were washed three times with PBS and incubated for 15 min with 0.1% trypan blue stain solution at room temperature. After washing three times with PBS, 0.1 N NaOH was added to the cells and trypan blue was quantified using the Victor quantitative detection spectrophotometer at 450 nm.

Measurement of Membrane Potential

Cells viability was also assessed using the luminescence-based kit CytoLite™ (Packard BioScience Co.) according to the recommendations of the manufacturer. Briefly, cells were cultured and treated in 96-well plates and after 72-hours incubation time, 25 μl of Activator solution was added to the cells followed by 150 μl of Amplifier solution. Luminescence was measured on a TopCount NXT™ counter (Packard BioSciences Co.) following a 5 minute precount delay.

Determination of Cellular ATP Levels

Cellular ATP concentrations were measured using the ATPLite-M™ luminescence assay (Packard BioSciences Co.). For this assay, cells were cultured on black 96-well ViewPlate™ and the ATP concentrations were measured on a TopCount NXT™ counter (Packard BioSciences Co.) following the recommendations of the manufacturer.

Radioimmunoassay

Progesterone production by MA-10 cells was measured by radioimmunoassay using anti-progesterone antisera (ICN, Costa Mesa, Calif.), following the conditions recommended by the manufacturer. The progesterone production was normalized by the amount of protein in each well. Radioimmunoassay data was analyzed using the MultiCalc software (EG&G Wallac, Gaithersburg, Md.).

22R-hydroxycholesterol-Protein Binding Blot Assay (CPBBA)

Purified Aβ (50 μM) and ³H-22R-hydroxycholesterol were incubated either alone or in the presence of 100 μM of unlabeled 22R-hydroxycholesterol (SP-222) or the various 22R-hydroxycholesterol derivatives in 20 μl volume for 8 or 24 h at 37° C. At the end of the incubation time, samples were separated by 1.5% agarose (Type I-B) gel electrophoresis under native conditions and transferred to nitrocellulose membrane (Schleicher & Schuell, Keene, N.H.) in 10×SSC buffer. The membrane was exposed to tritium-sensitive screen and analyzed by phosphoimaging using the Cyclone Storage phosphor system (Packard BioScience). Image-densitometric analysis was performed using the OptiQuant software (Packard BioScience). This method allows for the separation, visualization and identification of Aβ complexes, which have incorporated radiolabeled cholesterol (Yao Z. and Papadopoulos V., Function of β-amyloid in cholesterol transport: a lead to neurotoxicity, FASEB J 2002, 16:1677-1679), and 22R-hydroxycholesterol (Yao Z. X., et al., J Neurochem 2002, 83: 1110-1119), or 22R-hydroxycholesterol derivatives under native conditions. Low molecular weight unincorporated 22R-hydroxycholesterol and derivatives are separated and eliminated during electrophoresis.

Peptide Modeling and Docking Simulations

Computer docking of 22R-hydroxycholesterol and 16 of its derivatives with Aβ₁₋₄₂ was accomplished using an Aβ structure initialized by the solution structure of Aβ₁₋₄₀OMet(O) (MMDB Id: 7993 PDB Id: 1BA) resulting from data generated by CD and NMR spectroscopy. Watson A. A., Fairlie D. P. and Craik D. J., Solution structure of methionine-oxidized amyloid beta-peptide (1-40). Does oxidation affect conformational switching?, Biochem 1998, 37: 12700-12706. The Met(O) SME 35 residue was replaced by Met retaining the adjacent backbone dihedral angles and the I41 and A42 residues appended. The energy of the structure was then minimized using the Alchemy 2000 program (Tripos, St. Louis, Mo.). The 22R-hydroxycholesterol derivative structures were also generated using Alchemy 2000. Molecular docking was accomplished using Monte Carlo simulated annealing as previously described. Li H., Yao Z., Degenhardt B., Teper G. and Papadopoulos V., Cholesterol binding at the cholesterol recognition/interaction amino acid consensus (CRAC) of the peripheral-type benzodiazepine receptor and inhibition of steroidogenesis by an HIV TAT-CRAC peptide, Proc Natl Acad Sci USA 2001, 98: 1267-1272, implemented in modified versions of Autogrid/Autodock. Morris G. M., Goodsell D. S., Halliday R. S., Huey R., Hart W. E., Belew R. K. and Olson A. J., Distributed automated docking of flexible ligands to proteins: parallel applications of AutoDock 2.4, J Comput Chem 1998, 19: 1639-1662. For each of the compounds/Aβ pairs approximately 108 conformations were evaluated to obtain the selected one of minimum energy. Three sessions consisting of 100 runs, each starting at a random initial relative location and orientation of the ligand with respect to the target were executed. Each run was comprised of 100 annealing cycles using about 2×104 improvement steps. The average computation time for each ligand/target pair was about 2½ hours using a 1.7 GHz, 1 GB RAM PC.

Statistical Analysis

Statistical analysis was performed by one-way analysis of variance (ANOVA) and unpaired Student's t test using the INSTAT 3.00 (GraphPad, San Diego, Calif.).

Results

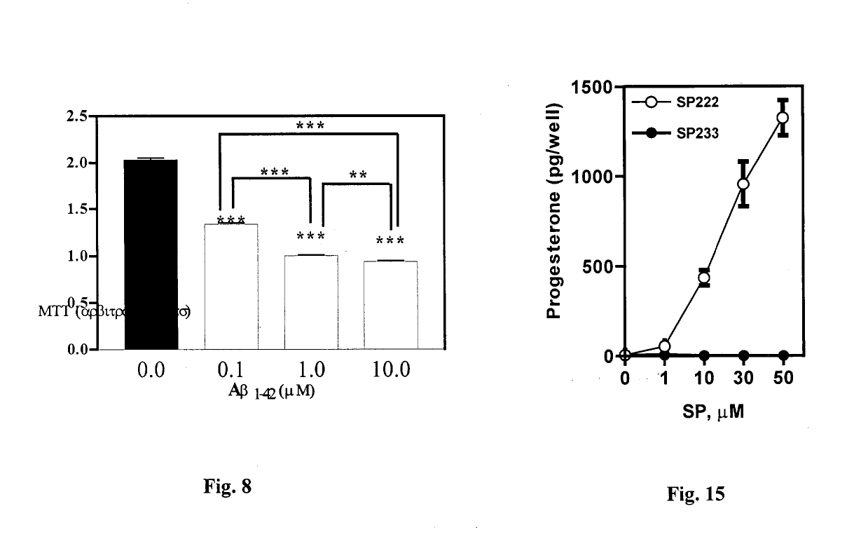

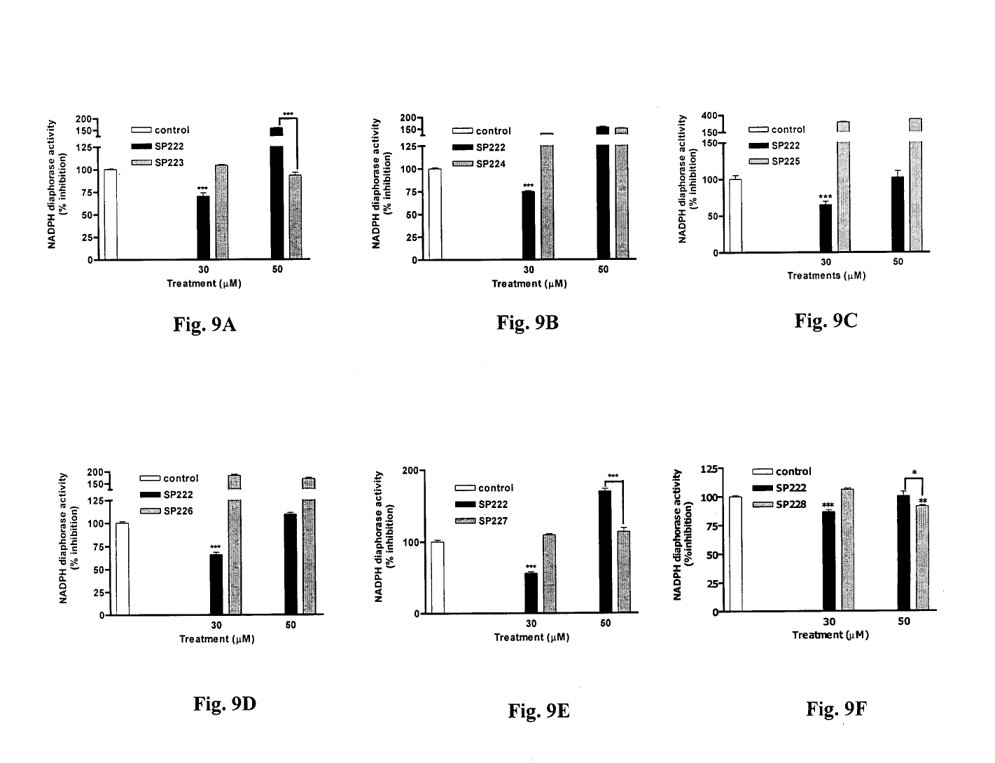

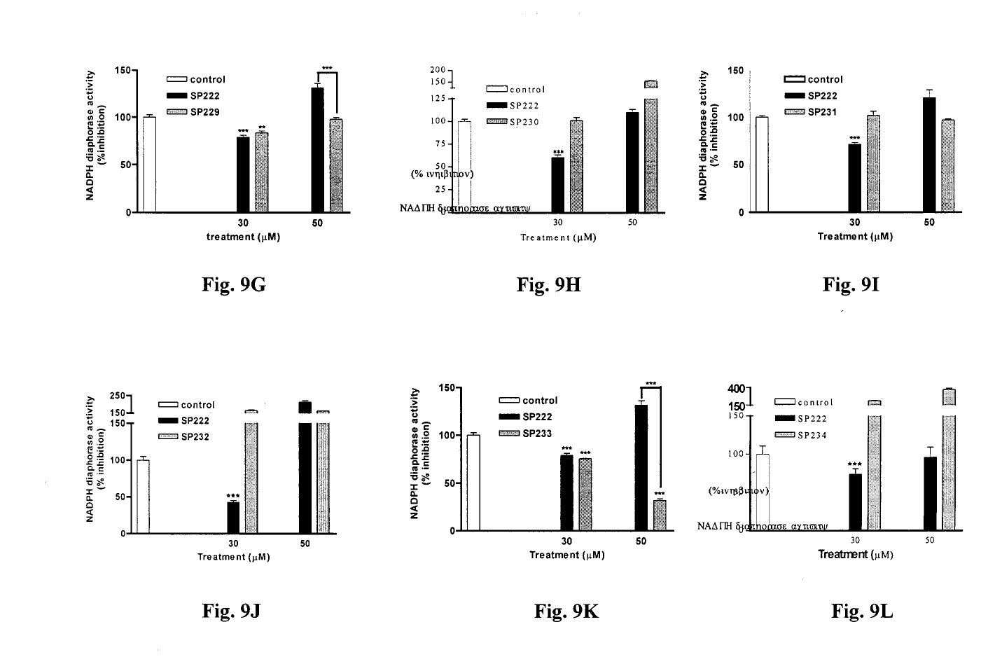

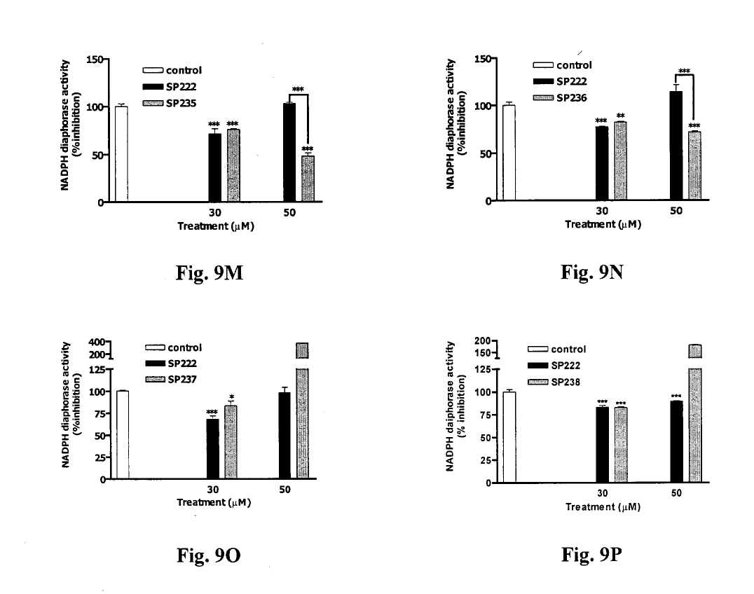

Three days exposure of PC12 cells to increasing concentrations of Aβ resulted in dose-dependent cell death (FIG. 8), reaching a maximum of 50% of the cells, in agreement with our previous data. Yao Z. X., et al., J Neurochem 2002, 83: 1110-1119; and Yao Z., Drieu K. and Papadopoulos V., The Gingko biloba extract EGb 761 rescues PC12 neuronal cells from β-amyloid-induced cell death by inhibiting the formation of β-amyloid-derived diffusible neurotoxic ligands, Brain Res 2001, 889:181-190. To stay close to the concentrations of Aβ present in AD brain, 0.1-10 μM concentrations of Aβ were used. The compounds tested for their neuroprotective properties were examined at 30 and 50 μM concentrations (FIGS. 9-15).

FIGS. 9-11 show the effect of the lead compound 22R-hydroxycholesterol (SP222) and the compounds containing the 22R-hydroxycholesterol structure (SP223-238) on Aβ-induced neurotoxicity determined using the MTT assay, a measurement of the NADPH diaphorase activity. FIGS. 9-11 show the effects of these compounds on 0.1, 1.0 and 10.0 μM Aβ-induced neurotoxicity, respectively, expressed as a percentage of inhibition of the NADPH diaphorase activity. The 100% inhibition level corresponds to the decrease of the blue formazan formation induced by Aβ administered alone.

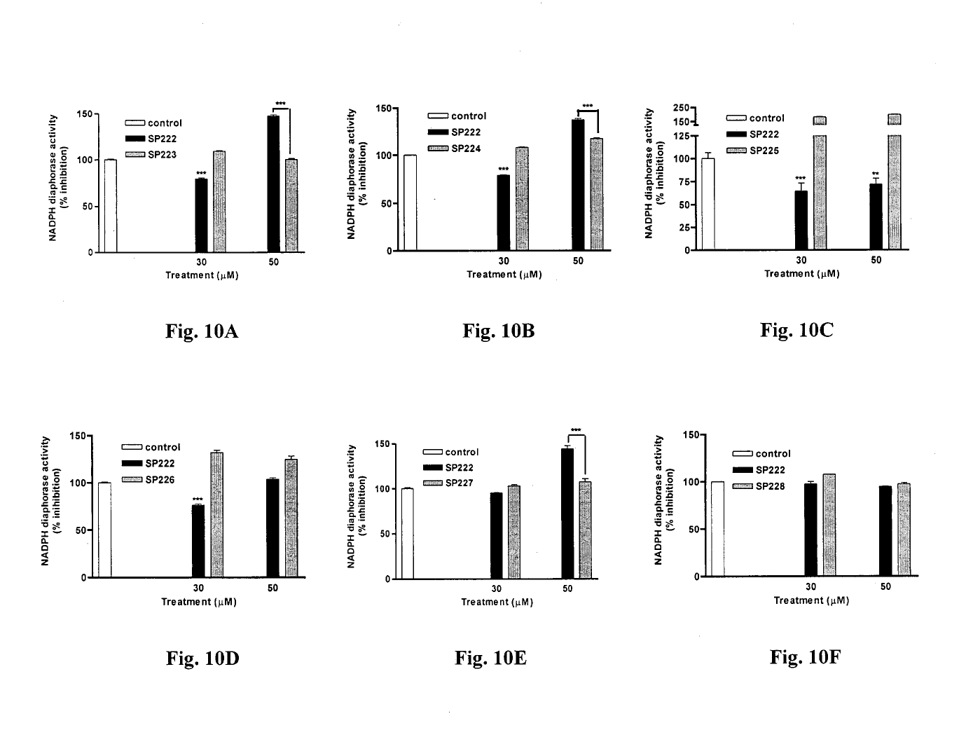

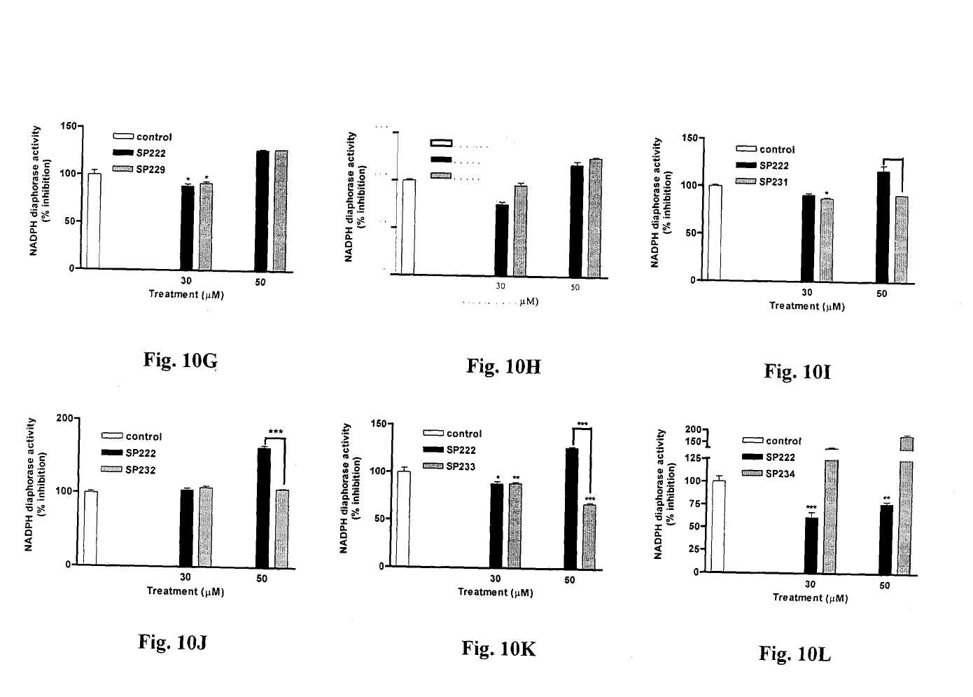

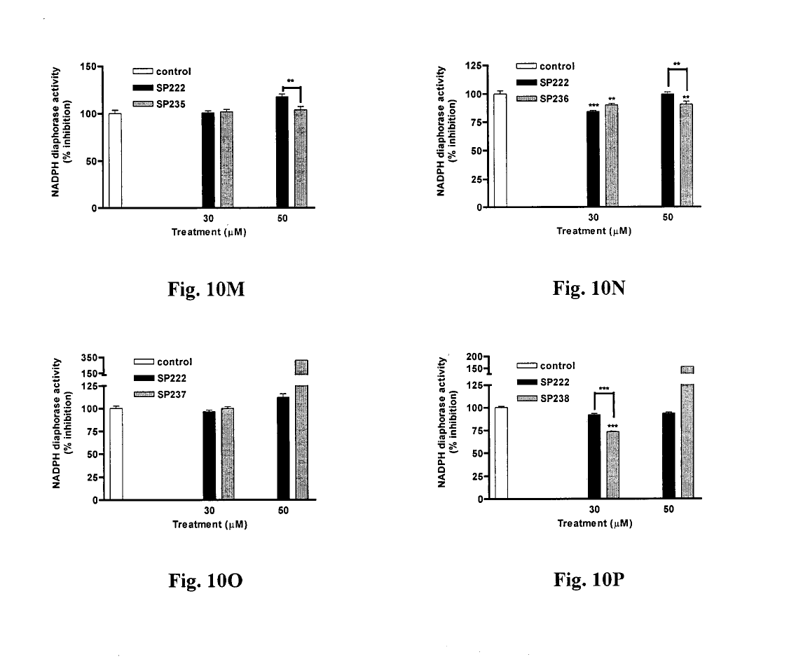

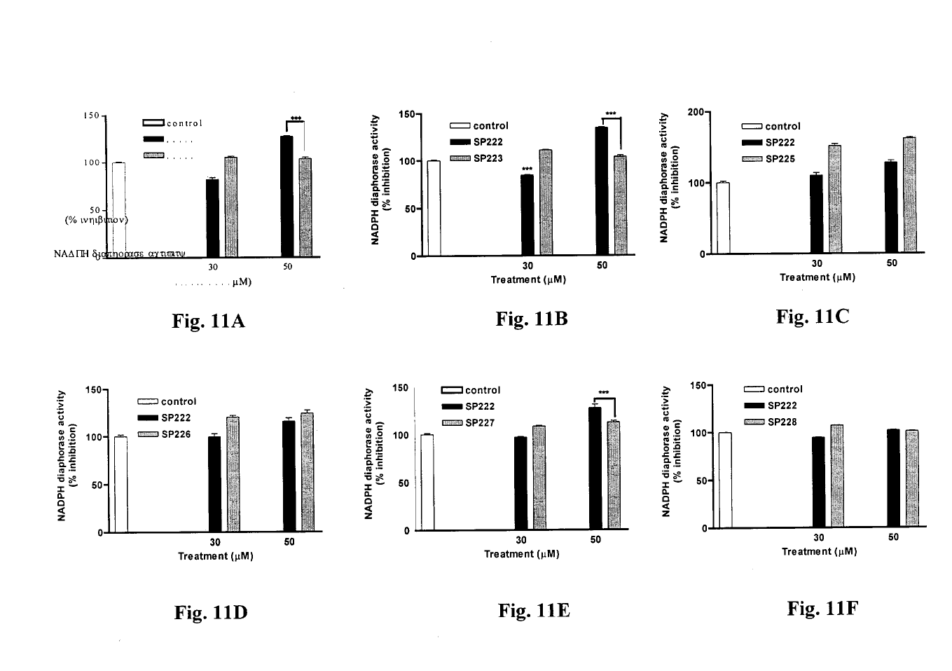

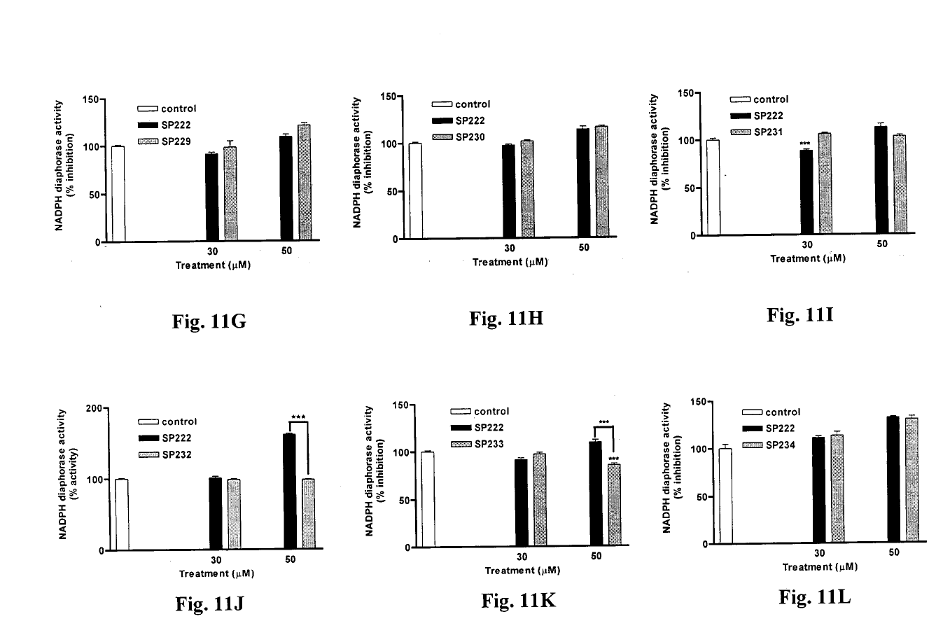

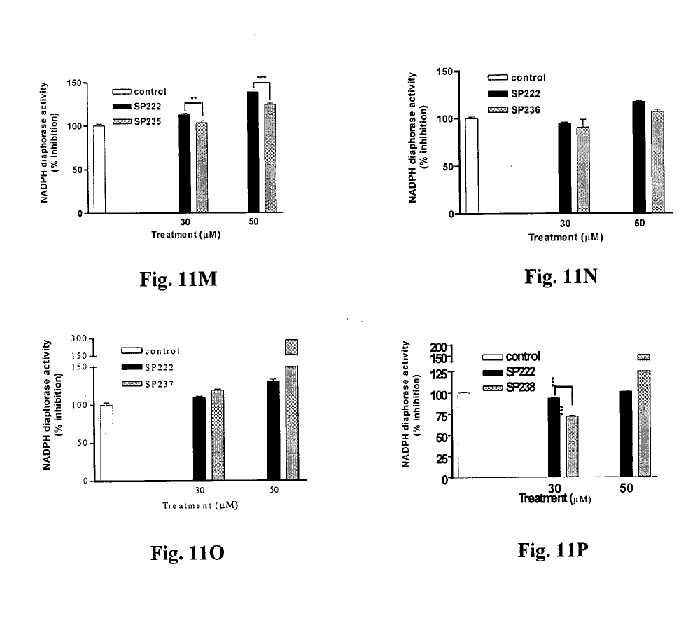

SP222 protects PC12 cells against Aβ 0.1 μM and 1 μM but provides a limited neuroprotection against Aβ given at 10 μM. It should be noted that a big variability was observed for the effect of SP-222 on high concentrations of Aβ, depending on the passage of the cells used. SP228, SP229, SP233, SP235, SP236, SP237 and SP238 displayed neuroprotective activity against Aβ 0.1 μM but only SP233, SP235, SP236 and SP238 exerted a significantly more robust effect than SP222 (FIGS. 9A-9P). SP233, SP236 and SP238 maintained their neuroprotective properties against 1 μM Aβ-induced toxicity (FIGS. 10A-10P) but only SP233 and SP238 kept this property in the presence of 10 μM Aβ (FIGS. 11A-11P).

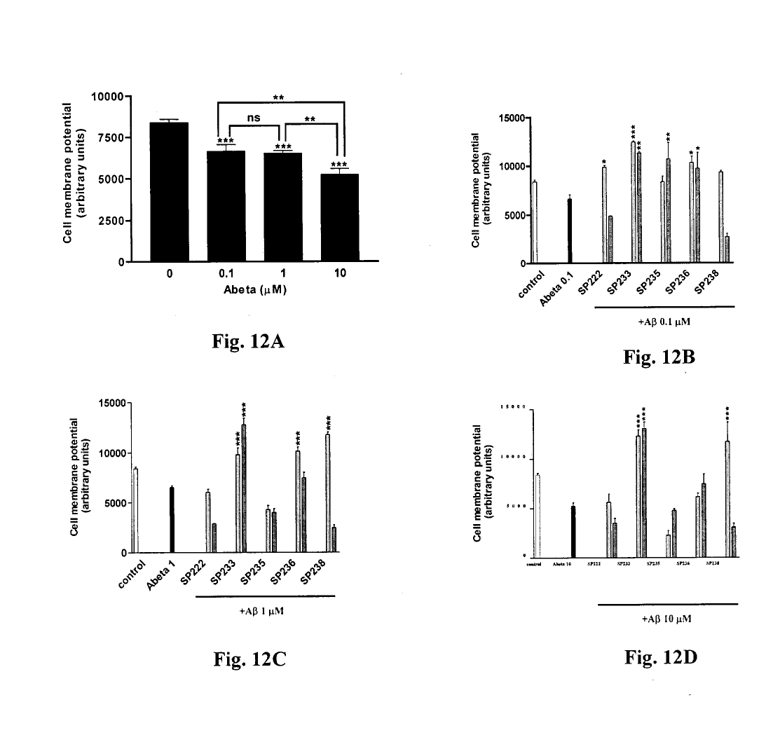

Results obtained with the MTT assay were confirmed using the membrane potential-assessing Cytolite assay for SP222, SP233, SP235, SP236 and SP238. FIG. 12A shows that Aβ exposure induces a dose-related decrease of the membrane potential-assessing luminescence. Although SP222 protected against 0.1 ∥M Aβ (FIG. 12B), it failed to do so against the two highest concentrations of Aβ (FIGS. 12C and 12D). The various SP compounds used displayed a significantly better neuroprotective effect compared to SP222 as shown by the increase in measured luminescence. The neuroprotective effect of SP233 and SP238 against 10 μM Aβ seen using the MTT assay (FIG. 1) was replicated by the raise of the signal under the same conditions (FIG. 12D).

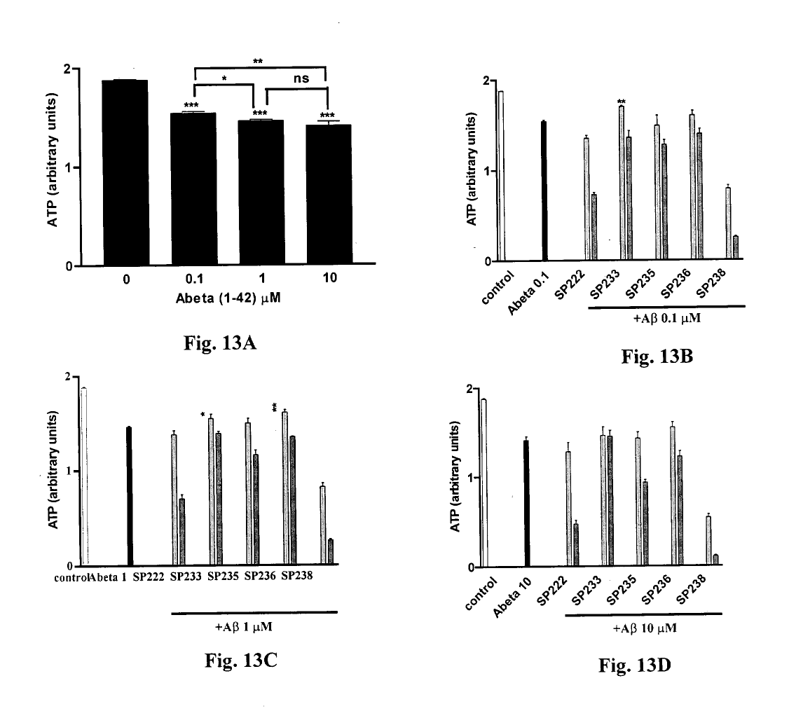

ATP levels, an index of mitochondrial function, were measured in PC12 cells treated with increasing concentrations of Aβ in the presence or absence of the SP222-SP238 compounds (FIGS. 13A-13D). Aβ decreased in a dose-dependent manner ATP production by PC12 cells; 18%, 22% and 25% decrease in ATP levels measured in the presence of 0.1, 1.0 and 10 μM Aβ, respectively (p<0.001 by ANOVA; FIG. 13A). From the compounds tested only SP233 and SP236 were able to reverse the 0.1 and 1.0 μM Aβ-induced decrease in ATP levels (FIGS. 13B and 13C). No beneficial effect of the SP compounds on ATP synthesis was seen in the presence of 10 μM Aβ.

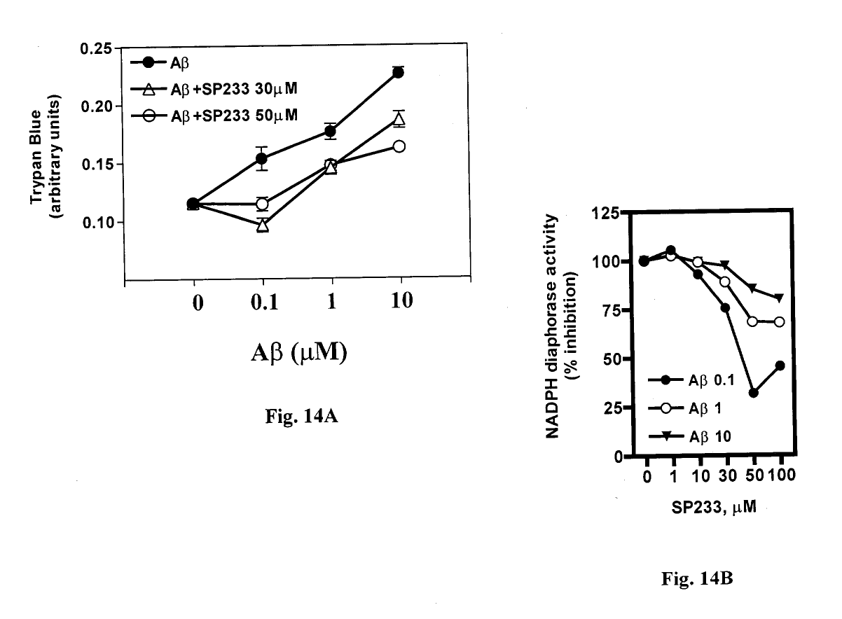

Trypan blue uptake by the cells was the fourth test used to assess the impact of the promising SP233 compound on Aβ-induced toxicity (FIG. 14A). As expected, 0.1, 1 and 10 M Aβ-induced a dose-dependent (33%, 36% and 97%, respectively; p<0.001 by ANOVA) increase in trypan blue uptake by PC12 cells. SP233 at 30 and 50 μM inhibited the Aβ-induced cell death (p<0.001 by ANOVA). FIG. 14B shows that the neuroprotective effect of SP233 is dose-dependent and it is maintained in the presence of all three concentrations of Aβ, although its efficacy decreases in presence of high, supra-physiopathological, Aβ concentrations.

One of the reasons in identifying 22R-hydroxycholesterol derivatives is the need of biologically active (neuroprotective) compounds that cannot be metabolized by P450scc to pregnenolone and then to tissue-specific final steroid products. To assess the metabolism of these compounds by steroidogenic cells we examined their ability to form steroids in MA-10 mouse tumor Leydig cells, a well-characterized steroidogenic cell model where 22R-hydroxycholesterol is an excellent P450scc substrate and can produce large amounts of steroids. Li H., Yao Z., Degenhardt B., Teper G. and Papadopoulos V., Cholesterol binding at the cholesterol recognition/interaction amino acid consensus (CRAC) of the peripheral-type benzodiazepine receptor and inhibition of steroidogenesis by an HIV TAT-CRAC peptide, Proc Natl Acad Sci USA 2001, 98: 1267-1272. FIG. 15 shows that in contrast to SP222, SP233 could not be metabolized to final steroid products.

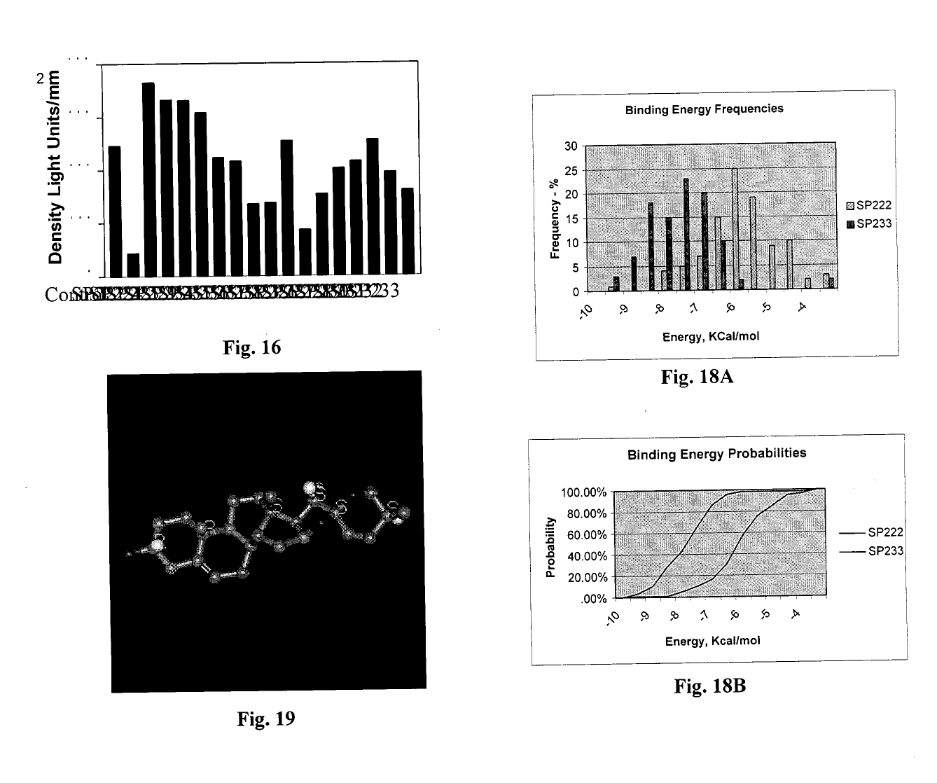

Considering the previous study on the mechanism underlying the neuroprotective action of 22R-hydroxycholesterol (SP222), where a direct interaction between 22R-hydroxycholesterol and Aβ was shown using the CPBBA method in Example 1, similar experiments were undertaken to investigate whether the 22R-hydroxycholesterol derivatives bind to Aβ. The direct interaction of these compounds to Aβ was shown in displacement studies performed against the radiolabeled 22R-hydroxycholesterol/Aβ complex (FIG. 16). Co-incubation of radiolabeled 22R-hydroxycholesterol together with Aβ for 24 hours at 37° C. demonstrated the presence of a high molecular weight radiolabeled band (FIG. 16) recognized by an antibody specific to Aβ (Yao et al., 2002 and data not shown). The specificity of radiolabeling of Aβ by 22R-hydroxycholesterol was demonstrated by competition studies using unlabeled 22R-hydroxycholesterol (FIG. 16) where 100 μM SP222 displaced by 80% radiolabeled SP222 compound bound to Aβ. From the SP compounds tested, SP237, SP238, SP226, SP227 and SP233 displaced radiolabeled 22R-hydroxycholesterol binding to Aβ by 46, 44, 65, 38 and 35%, respectively (FIG. 16).

These data were further confirmed using computational docking simulations with Aβ. The docking results show that Aβ₁₋₄₂ forms a pocket in the 19-36 amino acids area (FIG. 17) where 22R-hydroxycholesterol binds, in agreement with our previous data. Yao Z. X., et al., J Neurochem 2002, 83: 1110-1119. The docking energy for the various compounds tested placed in order of minimal energy required for binding to Aβ was: (−10.34 kcal/mol) SP229<SP232<SP224<SP237<SP222<SP233<SP228<SP223<SP230<SP234<SP225<SP238<SP236<SP226<SP235<SP231<SP227 (−8.35 kcal/mol). FIGS. 18A and 18B compare the binding characteristics of SP222 with SP233. This is an analysis of 100 docking runs with each of the compounds. The data shows that about 23% of the time SP233 docks with energy of −7.0 to −7.5 Kcal/mol while SP222 docks about 25% of the time with only 5.5 to 6.0 kcal/mol. The probability of SP233 having a stronger (more negative) docking energy is significantly greater than that for SP222. Almost 100% of the time SP233 binds with less than −6.0 kcal/mol while the equivalent number for SP222 is only about −4.0 kcal/mol. Analysis of the distribution of the binding energy frequencies indicates a bimodal profile suggesting the presence of two binding sites in Aβ. For SP233 peaks might be present at both −7 to −7.5 and −8 to −8.5 kcal/mol whereas with SP222 the peaks seem to be at −5.5 to −6.0 and −4.0 to −4.5 kcal/mol.

Discussion

Applicants finding that 22R-hydroxycholesterol declines in hippocampus and frontal cortex of AD brains compared to age-matched control specimens prompted the search of a function of this steroid in brain, leading to the finding that 22R-hydroxycholesterol protects rat PC12 and human differentiated NT2N cells against Aβ toxicity.

Applicants initially used the MTT assay, a widely used marker of cell viability and thus cytotoxicity. Using this assay, some of the compounds tested, namely SP233, SP235, SP236 and SP238, exhibited neuroprotective activity even when PC12 cells were exposed to concentrations as high as 10 μM Aβ. Interestingly, these compounds were more efficacious to the reference 22R-hydroxycholesterol (SP222) molecule.

A late event in the mechanism of action of Aβ is the direct or indirect disruption of the mitochondrial respiratory chain, leading to a decrease in ATP production that alone could lead to cell death. SP222, SP235, and SP238 compounds, which were able to rescue the PC12 cells from Aβ-induced toxicity, did not block the Aβ-induced changes in ATP synthesis. Although such an apparent discrepancy remains to be explained it is possible that the MTT assay (mitochondrial diaphorase activity) and ATP synthesis do not reflect the status of the same part of the respiratory chain. In contrast, SP233 and SP236 blocked, although in part, the Aβ-induced decrease in ATP production. The ability of SP233 to preserve ATP stocks could explain the potent neuroprotective effect of this compound, which was further confirmed by the trypan blue uptake cell viability assay. It should be noted that SP233 was found to be not only the most efficacious in all assays used but also the most potent, offering neuroprotection in vitro against Aβ at concentrations as low as 10 μM.

The studies presented herein were performed using 0.1, 1.0 and 10 μM Aβ₁₋₄₂. These concentrations are supra-physiopathological since the concentrations of Aβ₁₋₄₂ present in cerebrospinal fluid of AD patients and controls range from 500 1000 ng/l (0.1-0.2 nM). Even if Aβ₁₋₄₂ might be present in AD brain at 10 times higher concentration, the estimated pathophysiological concentrations of Aβ₁₋₄₂ would be in the range of 1-2 nM, which is 100-10,000 times less than the concentrations used in Applicants' experiments. With these considerations in mind it is clear that the 75% protection offered by SP233 against 0.1 μM Aβ is pharmacologically relevant.

Using the well-established MA-10 mouse Leydig cell model, Applicants demonstrated that unlike 22R-hydroxycholesterol, its bioactive derivative SP233 was unable to induce steroid formation.

The neuroprotective property of the SP compounds seems to follow a structure/activity relationship (SAR). SP231 and SP235 are stereoisomers of diosgenin (FIG. 1), but only SP235 is protective against Aβ-induced neurotoxicity. The stereochemistry of the SP235 is C3R, C10R, C13S, C20S, C22S, C25S, a motif shared by SP233 and SP236 (FIGS. 1 and 19). SP compounds exhibiting high neuroprotective activity and being active in the presence of high concentrations of Aβ contained an ester, preferably a fatty acid or a fatty acid-like structure, on C3. Indeed, SP235 that possesses an unsubstituted hydroxyl group in C3 offers limited neuroprotection acting only against 0.1 μM Aβ. In contrast, SP236 that is the succinic ester at C3 of SP235 is active against higher Aβ concentrations and SP233, which is a hexanoic ester at C3 of SP235 is the most potent compound. The finding that SP238 was able to protect PC12 cells against Aβ-induced toxicity, although it had no effect on maintaining ATP levels, further supports this hypothesis because its derivative without any side-chain on C3 (SP226) did not offer neuroprotection. The finding that benzoic acid substitution, present on SP232, was not effective in neuroprotection suggested that the presence of an aliphatic chain at this level is more relevant that an aromatic structure. Although these data are indicative of a SAR and highlights the importance of the presence of a fatty acid chain at C3, further modeling and SAR studies need to be performed to optimize the SP233 structure for neuroprotection.

Yao et al. recently demonstrated that the neuroprotective effect of the 22R-hydroxycholesterol lies in its ability to bind and inactivate Aβ₁₋₄₂. Based on this observation Applicants examined the ability of SP222 derivatives to offer neuroprotection by acting in a similar manner. Applicants' findings indicate that SP compounds exhibiting neuroprotective properties against Aβ-induced cell death displace radiolabeled 22R-hydroxycholesterol bound to the amyloid peptide.

Computational docking simulations were used to further characterize the SP-Aβ interaction. The studies revealed that two binding sites might be present on Aβ for the bioactive SP compounds. One binding site seems to be more specific for 22R-hydroxycholesterol (SP222), whereas the second binding site displays higher affinity for compounds such as SP233 and SP236. Although SP226 is shown to bind to this second binding site too, the calculated binding energy for this compound is much lower than the energy displayed by the neuroprotective SP molecules. A subsequent computational docking simulation study indicated that the binding energies of SP222 and SP233 follow a bimodal distribution, a finding that strongly supports the presence of two binding sites on Aβ. Further calculation of binding energies indicated that SP222 has less affinity for the second binding site compared to SP233 and suggests that the presence of the ester chain might be responsible for the ability of SP233 to bind to both sites on Aβ. Based on these observations Applicants hypothesize that occupancy of the Aβ second binding site might be required for a sustained inactivation of the amyloid peptide. Applicants are now in the process of testing this hypothesis in vitro and in silico.

Other mechanisms not related to a direct inactivation of Aβ could also contribute to the neuroprotective activity of SP233. A possible modulation of the steroid receptor family cannot be excluded although little is known about the binding of spirostenols on nuclear receptors. It has been shown that Aβ inhibits the fusion of GLUT3-containing vesicles leading to the disruption of mitochondrial homeostasis and, thus to neuronal death. On the other hand, the glucose absorption is enhanced in normal and streptozotocin-induced diabetic mice by spirostenol derivatives extracted from Polygonati rhizome. Taken together, these results suggest that restoration of glucose transport inside the cell might be a protective mechanism in our model activated by the spirostenol SP233. Natural and synthetic derivatives of diosgenin have been also shown to lower cholesterol absorption by the cell and to decrease cholesterol synthesis by inhibiting the key enzyme 3-hydroxy-3-methylglutaryl coenzyme A reductase. It is also well known that an increase of cellular cholesterol concentration induces the activation of β- and γ-secretase leading to Aβ production. Moreover, diosgenin derivatives have been shown to modify intracellular cholesterol pools by inhibiting the cholesteryl ester transfer protein, an enzyme reported to positively modulate the generation of Aβ. Although it is unlikely that these protective mechanisms take place in Applicants' model because they add Aβ in the culture medium, they could however be part of the in vivo response to SP233.

Despite the tremendous efforts undertaken during the past few years to discover novel therapeutic modalities for the cure and/or slowing of the progression of AD, no major clinical advances have been made since the introduction of acetylcholine-esterase inhibitors which are able to slow the progression of the disease in 10-15% of the patients and for a limited time period. Although many compounds are actually in clinical trials in an attempt to treat AD, for most of those AD pathology is a target secondary to their primary action. Such drugs include antioxidants, COX-1 and COX-2 inhibitors, statins, and brain vessel vasodilators. Applicants' results indicate that naturally occurring spirostenol compounds protect neuronal cells against Aβ.

A number of embodiments of the invention have been described. Nevertheless, it will be understood that various modifications may be made without departing from the spirit and scope of the invention. Accordingly, other embodiments are within the scope of the following claims, and as various changes can be made to the above compositions, formulations, combinations, and methods without departing from the scope of the invention, it is intended that all matter contained in the above description be interpreted as illustrative and not in a limiting sense. All patent documents and references listed herein are incorporated by reference.

What is claimed is:

1. A method of treating a neurodegenerative disorder in a subject in need thereof, comprising administering to the subject a compound of formula (I): 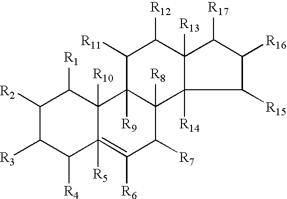
wherein each of R₁, R₂, R₄, R₅, R₆, R₇, R₁₁, R₁₂, R₁₅, and R₁₆, independently, is hydrogen, alkyl, hydroxy, amino, carboxyl, oxo, sulfonic acid, or alkyl that is optionally inserted with —NH—, —N(alkyl)-, —O—, —S—, —SO—, —SO₂—, —O—SO₂—, —SO₂—O—, —SO₃—O—, —CO—, —CO—O—, —O—CO—, —CO—NR′—, or —NR′—CO—; R₃ is a substituent as disclosed at R₃ of the compounds listed in Table 1 and FIG. 1; each of R₈, R₉, R₁₀, R₁₃, and R₁₄, independently, is hydrogen, alkyl, hydroxyalkyl, alkoxy, or hydroxy; and R17 is a substituent as disclosed at R₁₇ of the compounds listed in Table 1 and FIG. 1.

2. The method of claim 1 wherein the compound is selected from the group consisting of the compounds listed in Table 1.

3. The method of claim 1 wherein the compound is in a dosage form comprising a therapeutically effective amount of the compound.

4. The method of claim 3, wherein the dosage form is selected from the group consisting of tablet, soft gelatin capsule, hard gelatin capsule, suspension tablet, effervescent tablet, powder, effervescent powder, chewable tablet, solution, suspension, emulsion, cream, gel, patch, and suppository.

5. The method of claim 3, wherein the dosage form further comprises a pharmaceutically acceptable excipient.

6. The method of claim 5, wherein the pharmaceutically acceptable excipient comprises a binder, a disintegrant, a filler, a surfactant, a solubilizer, a stabilizer, a lubricant, a wetting agent, a diluent, an anti-adherent, a glidant, or a pharmaceutically compatible carrier.

7. The method of claim 1, further comprising administering at least one acetylcholinesterase inhibitor.

8. The method of claim 1, wherein the neurodegenerative disorder is selected from the group consisting of global and focal ischemic and hemorrhagic stroke, head trauma, spinal cord injury, hypoxia-induced nerve cell damage, nerve cell damage caused by cardiac arrest or neonatal distress, epilepsy, anxiety, diabetes mellitus, multiple sclerosis, phantom limb pain, causalgia, neuralgias, herpes zoster, spinal cord lesions, hyper algesia, allodynia, Alzheimer's Disease, Huntington's disease, and Parkinson's disease.

9. The method of claim 8, wherein the neurodegenerative disorder is Alzheimer's disease.

10. A pharmaceutical composition comprising a therapeutically effective amount of a compound of formula (I): 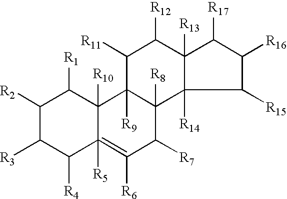
wherein each of R₁, R₂, R₄, R₅, R₆, R₇, R₁₁, R₁₂, R₁₅, and R₁₆, independently, is hydrogen, alkyl, hydroxy, amino, carboxyl, oxo, sulfonic acid, or alkyl that is optionally inserted with —NH—, —N(alkyl)-, —O—, —S—, —SO—, —SO₂—, —O—SO₂—, —SO₂—O—, —SO₃—O—, —CO—, —CO—O—, —O—CO—, —CO—NR′—, or —NR′—CO—; R₃ is a substituent as disclosed at R₃ of the compounds listed in Table 1 and FIG. 1; each of R₈, R₉, R₁₀, R₁₃, and R₁₄, independently, is hydrogen, alkyl, hydroxyalkyl, alkoxy, or hydroxy; and R17 is a substituent as disclosed at R₁₇ of the compounds listed in Table 1 and FIG. 1; and
a pharmaceutically acceptable excipient.

11. The pharmaceutical composition of claim 10, wherein the compound is selected from the group consisting of the compounds listed in Table 1.

12. The pharmaceutical composition of claim 10, wherein the pharmaceutical composition is in a dosage form selected from the group consisting of tablet, soft gelatin capsule, hard gelatin capsule, suspension tablet, effervescent tablet, powder, effervescent powder, chewable tablet, solution, suspension, emulsion, cream, gel, patch, and suppository.

13. The pharmaceutical composition of claim 10, wherein the pharmaceutically acceptable excipient comprises a binder, a disintegrant, a filler, a surfactant, a solubilizer, a stabilizer, a lubricant, a wetting agent, a diluent, an anti-adherent, a glidant, or a pharmaceutically compatible carrier.

14. The pharmaceutical composition of claim 10, further comprising at least one acetylcholinesterase inhibitor.

15. A method of identifying a compound having binding affinity to β-amyloid, comprising:
screening a database of known chemical compounds for structural homology to 22R-hydroxycholesterol;
ranking the compounds in the database based on a degree of homology to 22R-hydroxycholesterol;
extracting from the database compounds having a highest structural homology to 22R-hydroxycholesterol;
ranking the extracted compounds according to in vitro binding to β-amyloid; and
selecting the compound having the highest in vitro affinity.

16. A method of designing a compound having binding affinity to β-amyloid, comprising:
mapping 22R-hydroxycholesterol into two or more separate building blocks;
designing a new compound by modifying one or more blocks of 22R-hydroxycholesterol;
ranking the designed compound according to in vitro binding to β-amyloid; and
selecting the compound having the highest in vitro binding affinity.

17. A method of designing a compound having binding affinity to β-amyloid comprising:
mapping β-amyloid;
constructing on a computer screen a compound that complements the structure of β-amyloid or a fragment thereof;
ranking the constructed compound according to in vitro binding to β-amyloid; and
selecting the compound having the highest in vitro binding affinity.

18. The method of claim 17, wherein the fragment consists of amino acids 17 to 40 of β-amyloid.

19. The method of claim 17, wherein the fragment consists of amino acids 15 to 40 of β-amyloid.

20. The method of claim 17, wherein the fragment consists of amino acids 17 to 38 of β-amyloid.

21. The method of claim 17, wherein the fragment consists of amino acids 16 to 39 of β-amyloid.

22. A method of detection and quantification of Aβ in biological fluid, comprising:
obtaining a sample fluid;
incubating the fluid with a labeled compound of formula (I): 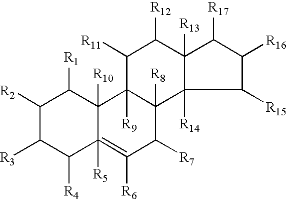
wherein each of R₁, R₂, R₄, R₅, R₆, R₇, R₁₁, R₁₂, R₁₅, and R₁₆, independently, is hydrogen, alkyl, hydroxy, amino, carboxyl, oxo, sulfonic acid, or alkyl that is optionally inserted with —NH—, —N(alkyl)-, —O—, —S—, —SO—, —SO₂—, —O—SO₂—, —SO₂—O—, —SO₃—O—, —CO—, —CO—O—, —O—CO—, —CO—NR′—, or —NR′—CO—; R₃ is a substituent as disclosed at R₃ of the compounds listed in Table 1 and FIG. 1; each of R₈, R₉, R₁₀, R₁₃, and R₁₄, independently, is hydrogen, alkyl, hydroxyalkyl, alkoxy, or hydroxy; and R17 is a substituent as disclosed at R₁₇ of the compounds listed in Table 1 and FIG. 1;
separating samples from the incubation fluid and transferring the samples to a nitrocellulose membrane;
exposing the membrane to a tritium-sensitive screen; and
analyzing the contents of the membrane.

23. The method of claim 22, wherein incubating the fluid with the labeled compound of formula (I) is in the presence of increasing concentrations of an unlabeled compound of formula (I).

24. The method of claim 22, wherein the step of analyzing the contents of the membrane comprises analyzing the contents of the membrane by at least one of phospho-imaging to detect the presence of Aβ and quantifying the amount of Aβ present in the biological fluid.

25. A method of diagnosing Alzheimer's disease in a subject, comprising:
obtaining a sample fluid from the brain of the subject;
incubating the fluid with a labeled compound of formula (I): 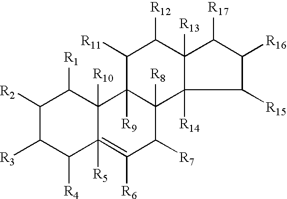
wherein each of R₁, R₂, R₄, R₅, R₆, R₇, R₁₁, R₁₂, R₁₅, and R₁₆, independently, is hydrogen, alkyl, hydroxy, amino, carboxyl, oxo, sulfonic acid, or alkyl that is optionally inserted with —NH—, —N(alkyl)-, —O—, —S—, —SO—, —SO₂—, —O—SO₂—, —SO₂—O—, —SO₃—O—, —CO—, —CO—O—, —O—CO—, —CO—NR′—, or —NR′—CO—; R₃ is a substituent as disclosed at R₃ of the compounds listed in Table 1 and FIG. 1; each of R₈, R₉, R₁₀, R₁₃, and R₁₄, independently, is hydrogen, alkyl, hydroxyalkyl, alkoxy, or hydroxy; and R17 is a substituent as disclosed at R₁₇ of the compounds listed in Table 1 and FIG. 1;
separating samples from the incubation fluid and transferring the samples to a nitrocellulose membrane;
exposing the membrane to a tritium-sensitive screen; and
analyzing the contents of the membrane.

26. The method of claim 25, wherein incubating the fluid with the labeled compound of formula (I) is in the presence of increasing concentrations of unlabeled compound of formula (I).

27. The method of claim 25, wherein the step of analyzing the contents of the membrane comprises analyzing the contents of the membrane by at least one of phospho-imaging to detect the presence of Aβ and quantifying the amount of Aβ present in the biological fluid.
